SEQUENCE LISTING (16 sequences)

SEQ ID NO: 1 - protein, 31 aa, Synthetic construct
TGERPFQCNQCGASFTQKGNLLRHIKLHSGE

SEQ ID NO: 2 - protein, 20 aa, synthetic construct
SLCCKQCQETEITTKNEIFS

SEQ ID NO: 3 - protein, 20 aa, synthetic construct
LSLCGPMAAYVNPHGYVHET

SEQ ID NO: 4 - protein, 20 aa, synthetic construct
LTVYKACNLNLIGRPSTEHS

SEQ ID NO: 5 - protein, 20 aa, synthetic construct
WFPGYAWTVAQCKICASHIG

SEQ ID NO: 6 - protein, 20 aa, synthetic construct
WKFTATKKDMSPQKFWGLTR

SEQ ID NO: 7 - protein, 9 aa, synthetic construct
SALLPTIPD

SEQ ID NO: 8 - DNA, 93 bp, synthetic construct
  source 1..93: mol_type = other DNA
actggagaacggcccttccagtgcaatcagtgcggggcctcattcacccagaagggcaacctgctccggcacatcaagctgcattccggggag

SEQ ID NO: 9 - DNA, 327 bp, synthetic construct
  source 1..327: mol_type = other DNA
tccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagac

SEQ ID NO: 10 - protein, 452 aa, Homo sapiens
MDFFRVVENQQPPATMPLNVSFTNMYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRMELKRSFFALRDQIPELENNEKAPKVVILKKATAYILSVQAEEQKLISEEDLLRKRREQLKHKLEQLRNSCA

SEQ ID NO: 11 - protein, 503 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMMINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 12 - protein, 400 aa, Homo sapiens
MNPASAPPPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGSPAQQHAHLRQQSYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQSTDSGLGLGCYSVPTTPEDFLSNVDEMDTGENAGQTPMNINPQQTRFPDFLDCLPGTNVDLGTLESEDLIPLFNDVESALNKSEPFLTWL

SEQ ID NO: 13 - protein, 1151 aa, synthetic construct
MGDTAPPQAPAGGLGGASGAGLLGGGSVTPRVHSAIVERLRARIAVCRQHHLSCEGRYERGRAESSDRERESTLQLLSLVQHGQGARKAGKHTKATATAATTTAPPPPPAAPPAASQAAATAAPPPPPDYHHHHQQHLLNSSNNGGSGGINGEQQPPASTPGDQMSALIALQGSLKRKQVVNLSPANSKRPNGFVDNSFLDIKRIRVGENLSAGQGGLQINNGQSQIMSGTLPMSQAPLRKTNTLPSHTHSPGNGLFNMGLKEVKKEPGETLSCSKHMDGQMTQENIFPNRYGDDPGEQLMDPELQELFNELTNISVPPMSDLELENMINATIKQDDPFNIDLGQQSQRSTPRPSLPMEKIVIKSEYSPGLTQGPSGSPQLRPPSAGPAFSMANSALSTSSPIPSVPQSQAQPQTGSGASRALPSWQEVSHAQQLKQIAANRQQHARMQQHQQQHQPTNWSALPSSAGPSPGPFGQEKIPSPSFGQQTFSPQSSPMPGVAGGSGQSKVMANYMYKAGPSAQGGHLDVLMQQKPQDLSRSFINNPHPAMEPRQGNTKPLFHFNSDQANQQMPSVLPSQNKPSLLHYTQQQQQQQQQQQQQQQQQQQQQQQQQQQQQQQQQQSSSAQQQQQQQSSSAQQQQQQQQQQQQQQQQQQQQQQQQQQQPSSQPAQSLPSQPLLRSPLPLQQKLLLQQMQNQPIAGMGYQVSQQQRQDQHSVVGQNTGPSPSPNPCSNPNTGSGYMNSQQSLLNQQLMGKKQTLQRQIMEQKQQLLLQQQMLADAEKIAPQDQINRHLSRPPPDYKDQRMVGNMQPTAQYSGGSSTISLNSNQALANPVSTHTILTPNSSLLSTSHGTRMPSLSTAVQNMGMYGNLPCNQPNTYSVTSGMNQLTQQMPKQLLANQNNPMMPRPPTLGPSNNNNVATFGAGSVGNSQQLRPNLTHSMASMPPQRTSNVMITSNTTAPNWASQEGTSKQQEALTSAGVRFPTGTPAAYTPNQSLQQAVGSQQFSQRAVAPPNQLTPAVQMRPMNQMSQTLNGQTMGPLRGLNLRPNQLSTQILPNLNQSGTGLNQSRTGINQPPSLTPSNFPSPNQSSRAFQGTDHSSDLAFDFLSQQNDNMGPALNSDADFIDSLLKTEPGNDDWMKDINLDEILGNNS

SEQ ID NO: 14 - protein, 100 aa, Homo sapiens
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHST

SEQ ID NO: 15 - protein, 112 aa, Homo sapiens
MGVEKQVIRPGNGPKPAPGQTVTVHCTGFGKDGDLSQKFWSTKDEGQKPFSFQIGKGAVIKGWDEGVIGMQIGEVARLRCSSDYAYGAGGFPAWGIQPNSVLDFEIEVLSVQ

SEQ ID NO: 16 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

(19) United States
(12) Patent Application Publication
(10) Pub. No.: US 2025/0314661 A1
(43) Pub. Date: Oct. 9, 2025

(54) CHEMIGENETIC TOOLS AND METHODS OF CONTROLLING AND ASSESSING PROTEIN PHASE SEPARATION

(71) Applicant: THE REGENTS OF THE UNIVERSITY OF CALIFORNIA, Oakland, CA (US)

(72) Inventor: Xiaokun SHU, San Francisco, CA (US)

(73) Assignee: THE REGENTS OF THE UNIVERSITY OF CALIFORNIA, Oakland, CA (US)

(21) Appl. No.: 18/866,298

(22) PCT Filed: May 15, 2023
(86) PCT No.: PCT/US2023/067016

§ 371 Date: Nov. 15, 2024

Related U.S. Application Data
(60) Provisional application No. 63/342,153, filed on May 15, 2022.

(51) Int. Cl. G01N 33/68; C07K 1/14

(52) U.S. Cl. CPC G01N 33/6845; C07K 1/14; G01N 2500/02

(57) ABSTRACT

The present disclosure is directed to compositions useful in, for example, screening for active compounds, inducing phase separation in a solution, and isolating a protein from a solution. Also disclosed are vaccines and compositions useful therein, which can be used to mutate an endogenous DNA sequence in a subject. This abstract is intended as a scanning tool for purposes of searching in the particular art and is not intended to be limiting of the present invention.

### CROSS-REFERENCE TO RELATED APPLICATIONS

This application claims priority to U.S. Provisional Ser. No. 63/342,153, filed May 15, 2022, which is incorporated by reference in its entirety.

### STATEMENT REGARDING FEDERALLY SPONSORED RESEARCH

This invention was made with government support under grants R01CA258327 and U01 DK127421 awarded by The National Institutes of Health. The government has certain rights in the invention.

### SUBMISSION OF SEQUENCE LISTING

The instant application contains a Sequence Listing which has been submitted electronically in XML format and is hereby incorporated by reference in its entirety. The name of the file containing the Sequence Listing is UCAL-025-PCT-SEQ LIST_ST26. The size of the text file is 20,456 bytes and the text file was created on May 15, 2023.

### FIELD

The disclosure relates to a system of amino acid sequences that, in the presence of an inducer, phase separate from a fluid into a solid or semi-solid granule, free of lipid membrane. The disclosure relates to using such granules as tools to separate, label and/or visualize protein-protein interactions that are unusually difficult to image at any resolution or to isolate and characterize because of their low concentration in solution.

### BACKGROUND

Protein-protein interactions (PPis) lead to the formation of protein complexes and machines (Alberts, B. (1998) Cell 92:291-294). At a larger spatial scale, macromolecular machines, proteins, and other biological molecules come together forming intracellular organelles. These structures are enveloped by lipid bilayer membranes that segregate and partially insulate their contents from the intracellular milieu. Recently, the importance of a different type of intracellular compartment that lack membranes has been recognized. Their size is intermediate between protein machines and intracellular organelles. Two major breakthroughs show a role for phase separation in their formation (Li et al. (2012) Nature 483:336-340; Brangwynne et al. (2009) Science 324:1729-1732).

Membraneless compartments, also known as biomolecular condensates, were first discovered more than a century ago (Banani et al. (2017) Nat Rev Mol Cell Biol, 1-14). However, molecular level physico-chemical mechanisms that lead to their formation remained unclear until recently. The first breakthrough came from the study of P granules, which are liquid condensates that form through liquid-liquid phase separation (LLPS) (Brangwynne et al. (2009) Science 324:1729-1732). Since then, LLPS has explained formation of a large number of biomolecular condensates from diverse types of biological molecules (Banani et al. (2017) Nat Rev Mol Cell Biol, 1-14; Shin and Brangwynne (2017) Science 357: eaaf4382; Bergeron-Sandoval et al. (2016) Cell 165:1067-1079; Hyman et al. (2014) Annu Rev Cell Dev Biol 30:39-58; Lyon et al. (2021) Nat Rev Mol Cell Biol, 1-21; Alberti et al. (2021) Nat ev Mol Cell Biol, 1-18). A key physical process driving protein phase separation is the multivalent interaction between the constituent macromolecules. The discovery that multivalent PPis can drive protein LLPS is the second breakthrough (Li et al. (2012) Nature 483:336-340). Multivalence is often introduced by folded multidomains in a protein, or by single folded domains that form oligomeric complexes, as well as by intrinsically disordered regions (IDRs) that contain short interaction motifs mediating weak and multivalent interactions.

New technologies that can manipulate protein condensates can help us gain a mechanistic understanding and appreciation of the functional roles of condensates in both normal cell physiology and disease states. Optogenetic tools were recently developed to induce protein droplet formation and have been useful in understanding biomolecular condensates (Shin et al. (2018) Cell 175:1481-1491; Sin et al. (2016) Cell, 1-28). Chemogenctic tools that can manipulate multivalent PPis and thus control protein LLPS arc another type of powerful technology that can be complementary to the optogenetic tools for this emerging field. Accordingly, there remains a need for chemogenetic tools that control protein liquid-liquid phase separation in living cells and methods of making and using same.

### SUMMARY

In accordance with the purpose(s) of the disclosure, as embodied and broadly described herein, the disclosure, in some embodiments, relates to compositions useful in, for example, screening for active compounds, inducing phase separation in a solution, and isolating a protein from a solution. Also disclosed are vaccines and compositions useful therein, which can be used to mutate an endogenous DNA sequence in a subject.

Thus, provided herein are compositions comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof. Also provided herein are cells comprising a disclosed composition. Also provided herein are methods of screening activity of plurality ocompounds, the method comprising: (a) exposing a compound to a composition comprising a first amino acid sequence and a second amino acid sequence for a time period sufficient to induce disassociation or association of the first amino acid sequence from or to, respectively the second amino acid sequence, wherein the first amino acid sequence comprises a zinc finger domain, or a functional fragment thereof; and wherein the second amino acid sequence comprises a cereblon (CRBN) amino acid domain, or a functional fragment thereof.

Also provided herein are methods of inducing phase separation in a solution, the solution comprising a composition comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof; the method comprising: (a) exposing a compound to a composition comprising a first amino acid sequence and a second amino acid sequence for a time period sufficient to induce disassociation or association of the first amino acid sequence and the second amino acid sequence.

Also provided herein are methods of isolating a protein or nucleic acid from a solution comprising: exposing a compound to a composition comprising a first amino acid sequence and a second amino acid sequence for a time period sufficient to induce disassociation or association of the first amino acid sequence and the second amino acid sequence; wherein the solution comprising a composition comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof. In some embodiments, the solution may be cytosol within a cell.

Also provided herein are vaccines comprising: a first amino acid sequence, a second amino acid sequence; and a third amino acid sequence or a nucleic acid sequence encoding a third amino acid sequence; wherein the first amino acid sequence comprises a zinc finger domain, or a functional fragment thereof; and wherein the second amino acid sequence comprises a cereblon (CRBN) amino acid domain, or a functional fragment thereof; wherein the third amino acid sequence comprises a tumor antigen (or pathogen antigen).

Also provided herein are compositions comprising: a first amino acid sequence, a second amino acid sequence; and a third amino acid sequence or a nucleic acid sequence encoding a third amino acid sequence; wherein the first amino acid sequence comprises a zinc finger domain, or a functional fragment thereof; and wherein the second amino acid sequence comprises a cereblon (CRBN) amino acid domain, or a functional fragment thereof at a molecular ratio from about 1 to about 1; and wherein the third aminoacid sequence or nucleic acid sequence encoding the third amino acid sequence is encapsulated within a particle comprising the first and second amino acid sequence; and wherein nucleic acid sequence encodes an enzyme or wherein the nucleic acid sequence is an sgRNA. Also provided herein are methods of mutating an endogenous DNA sequence in a subject comprising exposing a cell of a subject to a disclosed composition.

Also disclosed herein are methods of forming a particle in vivo or in vitro comprising: exposing a compound to a composition comprising a first amino acid sequence and a second amino acid sequence for a time period sufficient to induce disassociation or association of the first amino acid sequence and the second amino acid sequence; wherein the solution comprising a composition comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE FIGURES

The accompanying figures, which are incorporated in and constitute a part of this specification, illustrate several aspects and together with the description serve to explain the principles of the disclosure.

FIG. 1A shows a representative schematic illustrating the use of lenalidomide-inducible multivalent interaction of CEL & ZIF to drive condensate formation of a protein of interest (POI).

FIG. 1B shows a representative schematic illustrating the use of rapamycin-inducible recruitment of highly soluble protein, e.g., SUMO to dissolve condensate.

FIG. 2A-C show representative data illustrating the structure-based design of lenalidomide-inducible protein heterodimer for controlling protein-protein interaction. Specifically, FIG. 2A shows a structural model of lenalidomide-induced protein complex containing DDB1, cereblon (CRBN), and zinc finger 2 (ZF2) oflkaros (IKZF1), built by SWISS-model using the crystal structure of DDB1-CRBN-CK1a (pdb: 5fqd). The model illustrates design of lenalidomide-controllable protein heterodimer CEL (i.e., CBRNcm) and ZIF (i.e., IKZFIZF²). CRBNCTD: c-terminal domain of CRBN. IKZF1²_{F}², zinc finger 2 of IKZF1. FIG. 2B shows a Western blot against DDB1 after FLAG pull-down of CRBN or CEL. HEK293 cells were transfected with FLAG-tagged CRBN or CEL, in the absence or presence of exogenous DDB1 over-expression. Referring to FIG. 2C, a schematic of the translocation of SOScat from cytoplasm to plasma membrane via lenalidomide-induced interaction between CEL and ZIF is shown (left). The relocated SOScat then activates Ras, which leads to ERK activation via the MAPK pathway. The middle panel shows fluorescence images upon addition of 1 μM lenalidomide to HEK293 cells expressing CEL-IFP2-SOScat and ZIF-RFP-CAAX. Fluorescence images of HEK293 cells expressing ERK activity reporter ERK-SPARK, CEL-IFP2-SOScat and ZIF-RFP-CAAX upon addition of 1 μM lenalidomide are shown on the right. Scale, 10 μm.

FIG. 3A-G show representative data illustrating engineering of a lenalidomide-controllable hemogenetic tool for manipulating protein phase separation. Specifically, FIG. 3A (left) shows a schematic of lenalidomide-inducible and multivalent HOTag-based protein heterodimer for controlling protein phase separation. FIG. 3A (right) shows fluorescence images before and after addition of 1 μM lenalidomide to HEK293 cells expressing the two constructs shown in the left. FIG. 3B shows time-lapse images of HEK293 cells expressing SparkDrop (i.e., the two constructs of CEL-EGFP-HOTag3 and ZIF-EGFP-HOTag6) upon addition of 1 μM lenalidomide. FIG. 3C shows quantitative analysis of droplet formation over time after addition of lenalidomide or DMSO in HEK293 cells expressing SparkDrop or without CEL or ZIF. Error bar represents standard deviation (n=3). FIG. 3D (left panel) shows time-lapse images showing fusion events of two SparkDrops in of HEK293 cells. FIG. 3D (middle panel) shows the aspect ratio of two fusing droplets over time. FIG. 3D (right panel) shows the inverse capillary velocity averaged from seven fusion events. Error bar represents standard deviation. FIG. 3E shows the fluorescence recovery after photobleaching (FRAP) of the fluorescent droplets. FIG. 3F shows time-lapse images showing droplets disassembly after removal of lenalidomide. HEK293 cells were preincubated with 1 μM lenalidomide for 10 min. Time-lapse imaging started after lenalidomide removal. Time is in min: sec. Error bar represents standard deviation (n=3). FIG. 3G shows the titration curve of normalized SPARK signal in cells incubated with various concentrations of lenalidomide. Error bar represents standard deviation (n=3). Scale bar. FIG. 3A: 20 μm; FIG. 3B: 3 μm; FIG. 3D: 2 μm; FIG. 3E: 1 μm; FIG. 3F: 10 μm.

FIG. 4A-F show representative data illustrating that phase separation of SG scaffold protein G2BP1 can recruit client proteins but not vice versa. Specifically, SparkDrop-induced (no stress stimuli such as arsenite) phase separation of G3BP1 recruits FUS (FIG. 4A-C). FIG. 4A shows a schematic of the experimental design with observed results. FIG. 4B shows time-lapse fluorescence images before and after (30 min) addition of 100 nM rapamycin to HEK293 cells expressing SparkDrop-Frb (i.e., constructs of CEL-Frb-EGFP-HOTag3 and ZIF-EGFP-HOTag6), FKBP-IFP2-G3BP1, and FUS-mKO3. FIG. 4C shows a fluorescence intensity plot against distance (dashed lines in FIG. 4B). The cells were preincubated with 1 μM lenalidomide for 30 minutes. Referring to FIG. 4D-F, SparkDrop-induced (no stress stimuli such as arsenite) phase separation of FUS does not recruit G3BP1. FIG. 4D shows a schematic of the experimental design with observed results. FIG. 4E shows time-lapse fluorescence images before and after (30 min) addition of 100 nM rapamycin to HEK293 cells expressing SparkDrop-Frb (i.e., constructs of CEL-Frb-EGFP-HOTag3 and ZIF-EGFP-HOTag6), FUS-mKO3-FKBP, and IFP2-G3BP1. FIG. 4F shows a fluorescence intensity plot against distance (dashed lines in FIG. 4E). The cells were preincubated with 1 UM lenalidomide for 30 minutes. Scale bar: FIG. 4B and FIG. 4E, 10 μm.

FIG. 5A-J show representative data illustrating that LLPS of YAP recruits and compartmentalizes transcriptional machineries. Specifically, FIG. 5A shows a schematic of SparkDrop-based YAP LLPS via multivalent interaction. The interaction between the engineered protein heterodimer is induced by lenalidomide. Multivalency is introduced by the de novo designed HOTag6 that is a tetramer. FIG. 5B shows time-lapse images of representative cells expressing the SparkDrop-YAP upon addition of lenalidomide. FIG. 5C shows the normalized SPARK signal over time. Data are shown as mean±SD (n=3). −45 nuclei were analyzed for each sample. Lena and DMSO: cells expressing Spark Drop-YAP were incubated with lenalidomide and DMSO, respectively. Lena (No ZIF): cells expressing CEL-EGFP-Y AP (but not the ZIF construct) were incubated with lenalidomide. FIG. 5D shows multicolor images of cells expressing SparkDrop-YAP and mKO3 fused TEAD4 before and after addition of lenalidomide. FIG. 5E shows fluorescence intensity against position along the dashed line. The inset corresponds to the boxed area in FIG. 5D. FIG. 5F shows fluorescence images illustrating colocalization of SparkDrop-YAP condensates with MED1-mK03 condensates. Cells were incubated with lenalidomide to induce SparkDrop-Y AP condensates. FIG. 5G shows time-lapse images illustrating recruitment and compartmentalization of MEDI to the SparkDrop-Y AP condensates over time after addition of lenalidomide that induces SparkDrop-YAP condensates. FIG. 5H shows fluorescence intensity against position along the dashed line. The insets correspond to the boxed area in FIG. 5G. FIG. 51 shows fluorescence images of cells expressing SparkDrop-Y AP stained with antibodies against RNA Pol 11-SSP (RNAPII-SSP). FIG. SJ shows fluorescence intensity against position along the dashed line shown in FIG. 51. Scale bars: 10 μm (FIG. 5B and FIG. 51—left), 5 μm (FIG. 5D, FIG. 5F, and FIG. 5G), 2 μm (FIG. 51—insets).

FIG. 6A-D show representative images illustrating that the SparkDrop-Y AP condensates produce nascent RNAs and upregulate a direct target gene of Y AP. Specifically, FIG. 6A (upper) shows fluorescence images of EU-labeled cells that express SparkDrop-y AP. The cells were short labeled with EU for 1 hour. Arrows point to the induced YAP condensates and the colocalized nascent RNA droplets. Arrowhead points to nucleoli. The nucleus marked by asterisk was not transfected with SparkDrop-YAP. FIG. 6A (lower) shows fluorescence intensity against position shown by the white dashed line in upper panel. FIG. 6B (upper) shows fluorescence images of EU-labeled cells that express SparkDrop without YAP. FIG. 6B (lower) shows fluorescence intensity against position shown by the white dashed line in upper panel. FIG. 6C shows a schematic showing experimental procedure for the RT-qPCR data shown in FIG. 6D. FIG. 6D shows normalized mRNA levels of Ctgf, a direct target gene of YAP. Data are shown as meant SD (n=3).

FIG. 7A and FIG. 7B show representative data illustrating that MYCN condensates can be dissolved by rapamycin-inducible recruitment of the highly soluble protein SUMO. Specifically. FIG. 7A shows representative fluorescence images. FIG. 7B shows quantitative analysis. Scale bars, 5 μm.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

The present disclosure can be understood more readily by reference to the following detailed description of the disclosure and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting. Although any methods and materials similar or equivalent to those described herein can be used in the practice or testing of the present invention, example methods and materials are now described.

While embodiments of the present invention can be described and claimed in a particular statutory class, such as the system statutory class, this is for convenience only and one of skill in the art will understand that each embodiment of the present invention can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### Definitions

Listed below are definitions of various terms used to describe this invention. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A): in yet another embodiment, to both A and B (optionally including other elements); etc.

The term “or” as used herein shall only be interpreted as indicating exclusive alternatives (i.e., “one or the other but not both”) when preceded by terms of exclusivity, “either,” “one of,” “only one of,” or “exactly one of.”

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), arc inclusive or open-ended and do not exclude additional, unrecited elements or method steps.

The term “about” as used herein when referring to a measurable value such as an amount,
a temporal duration, and the like, is meant to encompass variations of ±20%, ±10%, ±5%, ±1%, ±0.5%, or ±0.1% from the specified value, as such variations are appropriate to perform the disclosed methods.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to“A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by dimerization or association of the first and second amino acid sequences. For example, in the context of dimerization, such stimulation entails the association of the first amino acid domain with the second amino acid domain that results in a subsequent signal transduction even or activation event. Further, the stimulation event may activate a cell and upregulate or downregulate expression or secretion of a molecule, such as an RNA sequence or molecule. Thus, ligation of the amino acid sequences of the disclosure, even in the presence or absence of an inducer, may result in the reorganization of cytoskeletal structures, or in the coalescing of cell surface moieties, each of which could serve to enhance, modify, or alter subsequent cellular responses. In some embodiments, the activation event is the formation of a granules comprise the first and second amino acid domains. The abbreviations used herein have their conventional meaning within the chemical and biological arts. The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

The terms “cancer” and “cancerous” as used herein refer to or describe a physiological condition in mammals in which a population of cells are characterized by unregulated cell growth. Thus, the term “cancer” refers to a group of diseases involving abnormal cell growth with the potential to invade or spread to other parts of the body. Examples of cancer include, but not limited to, lung cancer, bone cancer, blood cancer, chronic myelomonocytic leukemia (CMML), bile duct cancer, cervical cancer, liver cancer, pancreatic cancer, skin cancer, cancer of the head and neck, cancer of the eye, cutaneous or intraocular melanoma, uterine cancer, ovarian cancer, rectal cancer, cancer of the anal region, stomach cancer, colon cancer, breast cancer, testicular cancer, gynecologic tumors (e.g., uterine sarcomas, carcinoma of the fallopian tubes, carcinoma of the endometrium, carcinoma of the cervix, carcinoma of the vagina or carcinoma of the vulva). Hodgkin's disease, cancer of the esophagus, cancer of the small intestine, cancer of the endocrine system (e.g., cancer of the thyroid, parathyroid or adrenal glands),

By “fragment” is meant a portion of a polypeptide or nucleic acid molecule, such as but not limiting to a truncation mutant. This portion contains, preferably, at least about 5%, 10%, 20%, 30%, 40%, 50%, 60%, 70%, 80%, 90%, 95% of the entire length of the reference nucleic acid molecule or polypeptide. A fragment may contain about 5, about 10, about 20, about 30, about 40, about 50, about 60, about 70, about 80, about 90, about 100, about 200, about 300, about 400, about 500, about 600, about 700, about 800, about 900, or about 1000 or more nucleotides or amino acids of a nucleotide or amino acid sequence, respectively.

The term “functional fragment” means any portion or fragment of a polypeptide or nucleic acid sequence from which the respective full-length polypeptide or nucleic acid relates that is of a sufficient length and has a sufficient structure to confer a biological affect that is similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain or comprise about 99%, about 98%, about 97%, about 96%, about 95%, about 94%, about 93%, about 92%, about 91%, or about 90% sequence identity to the wild-type or given sequence upon which the sequence is derived. In some embodiments, the functional fragment may retain about 85%, about 80%, about 75%, about 70%, about 65%, or about 60% sequence homology to the wild-type sequence upon which the sequence is derived.

As used herein, the term “genetic construct” is meant to refer to the DNA or RNA molecules that comprise a nucleotide sequence that encodes protein. The coding sequence includes initiation and termination signals operably linked to regulatory elements including a promoter and polyadenylation signal capable of directing expression in the cells of the individual to whom the nucleic acid molecule is administered.

The term “granule” means a solid or semi-solid phase of protein condensate. In some embodiments, the granule comprises a solid or semisolid matrix comprising the amino acid sequences disclosed herein. In some embodiments, the granule comprises the granule comprises a solid or semisolid matrix comprising the amino acid sequences disclosed herein and one or a plurality of target amino acid sequences or target nucleic acid molecules precipitated with the phase change from liquid phase to solid or semi-solid phase. In some embodiments, the granule comprises a solid or semisolid matrix comprising the amino acid sequences disclosed herein associated or bound to a target amino acid.

The term “host cell” as used herein is meant to refer to a cell that can be used to express a nucleic acid, e.g., a nucleic acid of the disclosure. The host cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated” as used herein means that the polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptide of the disclosure.

The terms “in isolation” mean that, for purposes of this disclosure, the nucleic acid may not be the species listed. In other words, the nucleic acid may incorporate the mutations above in combination with one or more other mutations listed or not listed, but the nucleic acid may not be defined as the single species containing the nucleic acid mutations listed.

The term “inverse capillary velocity” or “inversion capillary velocity” as used herein is a measurement of viscosity over surface tension of the droplet or granule. (n/y: here y is surface tension of the droplet; n is viscosity). In some embodiments the average inversion capillary velocity is from about 3.0 to about 3.1. In some embodiments, the average inversion capillary velocity if calculated and is from about 2.9 to about 3.2. In some embodiments, the average inversion capillary velocity if calculated and is from about 2.8 to about 3.3.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

The term “pharmaceutically acceptable salt” of tumor specific neoantigens as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic. 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is from about Oto about 4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Disease, disorder, and condition are used interchangeably herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

The term “inducer” means any molecule that facilitates, causes or triggers a precipitation (a polymerization or dimerization) event or a depolymerization or dissolution event in the presence of a first and a second amino acid sequence. In some embodiments, precipitation is the dimerization or granule formation of the first and second amino acid sequences. In some embodiments, the dissolution or a depolymerization event (in some embodiments, dissociation of the first and second amino acid domains resulting in dissolution of the granule).

The term “signaling pathway” as used herein refers to a series of interactions between cellular and optionally extra-cellular components (e.g., proteins, nucleic acids, small molecules, ions, lipids) that convey a change in one component to one or more other components, which in turn may convey a change to additional components, which is optionally propagated to other signaling pathway components.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A nucleic acid will generally contain phosphodiester bonds, although, in some embodiments, nucleic acid analogs may be included that may have at least one different linkage, e.g., phosphoramidate, phosphorothioate, phosphorodithioate, or 0-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR. NH.sub.2, NHR, N.sub.2 or CN, wherein R is C.sub. 1-C.sub.6 alkyl, alkenyl or alkynyl and halo is F, Cl. Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in US20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the nucleotide sequence encoding one or more antigens is free of modified nucleotide analogs. In some embodiments, the nucleotide sequence encoding one or more antigens comprises from about 1 to about 20 nucleic acid modifications. In some embodiments, the nucleotide sequence encoding one or more antigens comprises from about 1 to about 50 nucleic acid modifications. In some embodiments, the nucleotide sequence encoding one or more antigens independently comprise from about 1 to about 100 nucleic acid modifications.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also is a plasmid comprising one or more nucleotide sequences that encode one or a plurality of amino acid domains disclosed herein. In some embodiments, the disclosure relates to a host cell comprising a nucleic acid molecule encoding a first amino acid sequence, a second amino acid sequence or both the first and second amino acid sequences disclosed herein.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “animal” includes, but is not limited to, humans and non-human vertebrates such as wild, domestic, and farm animals.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, “conservative” amino acid substitutions may be defined as set out in Tables A, B, or C below. The compositions, pharmaceutical compositions and method may comprise nucleic acid sequences comprising or encoding amino acids sequences comprising one or more conservative substitutions. In some embodiments, the vaccines, compositions, pharmaceutical compositions and methods comprise nucleic acid sequences that retain from about 70% sequence identity to about 99% sequences identity to the sequence identification numbers disclosed herein but comprise one or more conservative substitutions. Conservative substitutions of the present disclosure include those wherein conservative substitutions (from either nucleic acid or amino acid sequences) have been introduced by modification of polynucleotides encoding polypeptides. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has similar binding affinities to its target. Exemplary conservative substitutions are set out in Table A

TABLE A
Conservative Substitutions I

| Side Chain Characteristics | Amino Acid |
|---|---|
| Aliphatic | |
| Non-polar | GAPILVF |
| Polar-uncharged | CSTMNQ |
| Polar-charged | DEKR |
| Aromatic | HFWY |
| Other | NODE |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic) | |
| Aliphatic: | ALIVP |
| Aromatic: | FWY |
| Sulfur-containing: | M |
| Borderline: | GY |
| Uncharged-polar | |
| Hydroxyl: | STY |
| Amides: | NQ |
| Sulfhydryl: | C |
| Borderline: | GY |
| Charged (Basic): | KRH |
| Charged (Acidic): | DE |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys(C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | ArgHis |
| Met(M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the amino acids described herein are intended to include nucleic acids and, where the inhibitors include polypeptide, polypeptides bearing one or more insertions, deletions, or substitutions, or any combination thereof, of amino acid residues as well as modifications other than insertions, deletions, or substitutions of amino acid residues.

As used herein, “more than one” or “two or more” of the aforementioned amino acid substitutions means 2, 3, 4, 5, 6, 7, 8, 9, 10 or more of the recited amino acid or nucleic acid substitutions. In some embodiments, “more than one” means 2, 3, 4, or 5 of the recited amino acid substitutions or nucleic acid substitutions. In some embodiments, “more than one” means 2, 3, 4 or more of the recited amino acid substitutions or nucleic acid substitutions. In some embodiments, “more than one” means 2, 3 or 4 of the recited amino acid substitutions or nucleic acid substitutions. In some embodiments, “more than one” means 2 or more of the recited amino acid substitutions or nucleic acid substitutions. In some embodiments, “more than one” means 2 of the recited amino acid substitutions or nucleic acid substitutions.

The “percent identity” or “percent homology” are used interchangeably of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences arc of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length within a query sequence that either match or satisfy some positive-valued threshold score T when aligned with a word of the same length in a database sequence. T is referred to as the neighborhood word score threshold (Altschul et al., 1997). These initial neighborhood word hits act as seeds for initiating searches to find HSPs containing them The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

As used herein, the terms “subject,” “individual,” “host,” and “patient.” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human.

As used herein. “patient in need thereof” or “subject in need thereof” refers to a living organism suffering from or prone to a disease or condition that can be treated by administration of at least one composition, vaccine or pharmaceutical composition disclosed herein, including, for example, a vaccine comprising a nucleic acid sequence encoding an amino acid sequence or composition disclosed herein or a vaccine comprising a granule disclosed herein, such as a nucleic acid sequence that encodes a first and second amino acid domain according to the methods described herein. A “patient in need thereof” or “subject in need” may also refer to a living organism that is receiving vaccine (or pharmaceutical composition comprising a vaccine), or has received a vaccine (or pharmaceutical composition comprising a vaccine); or has a tumor or cancer. Non-limiting examples include humans, other mammals, such as bovines, rats, mice, dogs, monkeys, horse, cats, goat, sheep, cows, deer, and other non-mammalian animals. In embodiments, a patient in need thereof or subject in need thereof is human. In some embodiments, the subject in need thereof is a human patient that is suspected of having cancer or has been diagnosed with cancer and exhibits.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It should be noted that any embodiment of the disclosure can optionally exclude one or more embodiment for purposes of claiming the subject matter.

### Compositions and Systems

Provided herein are compositions comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof.

Also provided herein are compositions comprising: a first amino acid sequence, a second amino acid sequence; and a third amino acid sequence or a nucleic acid sequence encoding a third amino acid sequence; wherein the first amino acid sequence comprises a zinc finger domain, or a functional fragment thereof; and wherein the second amino acid sequence comprises a cereblon (CRBN) amino acid domain, or a functional fragment thereof at a molecular ratio from about 1 to about 1; and wherein the third amino acid sequence or nucleic acid sequence encoding the third amino acid sequence is encapsulated within a particle comprising the first and second amino acid sequence; and wherein nucleic acid sequence encodes an enzyme or wherein the nucleic acid sequence is an sgRNA. In some embodiments, the disclosure relates to a nucleic acid sequence encoding a fusion protein, the fusion protein comprising a first and second domain, wherein the first domain comprising the first amino acid sequence that is a zinc finger domain and the second domain comprises a second amino acid sequence comprising a CRBN domain. In some embodiments, the nucleic acid sequence encoding the first domain is on a single nucleic acid molecule and the nucleic acid sequence encoding the second domain is on a second nucleic acid molecule. In some embodiments, a third nucleic acid sequence encodes a target protein. The third nucleic acid encoding a target protein can encode an amino acid within the first or second domain such that the first or second domain comprises a fusion protein between the zinc finger domain and the target protein or a fusion protein between the CRBN amino acid domain and the target protein. In some embodiments, a single nucleic acid molecule comprises a first nucleic acid sequence encoding a zinc finger amino acid domain, a second nucleic acid encoding a CRBN amino acid domain and third nucleic acid domain encoding a target protein. In some embodiments, the fusion protein folds upon exposure to an inducer or condensing agent, such that the CRBN amino acid domain and the zinc finger domain associate, create a phase change and precipitate the target protein in the new phase. Such phase change provides an option to isolate or manipulate the target protein in the lab when isolation of the target protein within the first and second domain is not possible. Additionally, the systems and compositions disclosed herein can also be useful for precipitating portions of cells that become trapped in the group of particles formed after phase separation of the amino acids disclosed herein. In such embodiments, scientists may isolate or manipulate or identify unknown proteins captured in the particle. This is especially useful when the cell involved in the phase separation is a diseased cell, such as a cancer cell. In such embodiments, the phase separation can be used as a discovery platform around which a particle comprising the first and second amino acid domains comprises one or a plurality of target proteins. After isolation of the contents of the particles or the proteins that are expressed by the first and second amino acid domains, one of ordinary skill may identify the target protein by sequencing the amino acid sequence of the target protein or exposing the target protein to an antibody or antibody fragment that is known to bind or associate with an amino acid sequence. Binding of the antibody or antibody fragment or sequencing the amino acid sequence of the target protein can reveal its identity. The disclosure relates to cloning or subcloning nucleic acid sequences encoding the zinc finger component into a nucleic acid molecule (e.g. a nucleic acid plasmid or cosmid), such that the amino acid sequences of the disclosure may be expressed. In some embodiments cells express the following sequences or are transformed or transduced with nucleic acid sequences encoding the following sequences.

Zinc Finger Domain
ZIF DNA sequence
(SEQ ID NO: 8)
actggagaacggcccttccagtgcaatcagtgcggggcctcattcacccagaagggcaacctgctceggcacatcaagctgcattcc
ggggag
Zinc Finger (ZIF) Amino Acid Sequence
(SEQ ID NO: 1)
TGERPFQCNQCGASFTQKGNLLRHIKLHSGE
CRBN Domain
CEL nucleic acid sequence
(SEQ ID NO: 9)
tccctttgctgtaaacaatgtcaagaaacagaaataacaaccaaaaatgaaatattcagtttatccttatgtgggccgatggcagcttatgt
gaatcctcatggatatgtgcatgagacacttactgtgtataaggcttgcaacttgaatctgataggccggccttctacagaacacagctg
gtttcctgggtatgcctggactgttgcccagtgtaagatctgtgcaagccatattggatggaagtttacggccaccaaaaaagacatgtc
acctcaaaaattttggggcttaacgcgatctgctctgttgcccacgatcccagac
CEL amino acid sequences
(SEQ ID NO: 2)
SLCCKQCQETEITTKNEIFS
(SEQ ID NO: 3)
LSLCGPMAAYVNPHGYVHET
(SEQ ID NO: 4)
LTVYKACNLNLIGRPSTEHS
(SEQ ID NO: 5)
WFPGYAWTVAQCKICASHIG
(SEQ ID NO: 6)
WKFTATKKDMSPQKFWGLTR
(SEQ ID NO: 7)
SALLPTIPD

In some embodiments, the CRBN domain comprises SEQ ID NO:2, 3, 4, 5, 6, and 7 in a contiguous amino acid sequence, or any functional fragment of the aforementioned sequences in one contiguous sequence, wherein the functional fragments comprise at least about 75%, 85%, 90%, 92%, 93%, 94%, 95%, 96%, 97% or 99% sequence identity to any of the SEQ ID Nos disclosed above, or the nucleic acid sequences that encode the same.

In various aspects, the zinc finger is IZF1, ZIF or a functional fragment thereof. In various aspects, the zinc finger amino acid domain comprises at least about 70%, at least about 75%, at least about 80%, at least about 85%, or at least about 90% sequence identity to SEQ ID NO:1. In some embodiments, the zinc finger amino acid domain comprises at least about 91%, 92%, 93%, 94%, 95%, 96%, 97%, 98%, 99%, or 100% sequence identity to SEQ ID NO:1. In some aspects, the zinc finger amino acid domain comprises at least about 85% sequence identity to SEQ ID: NO: 1.

In various aspects, the CRBN amino acid domain comprises at least about 70%, at least about 75%, at least about 80%, at least about 85%, or at least about 90% sequence identity to SEQ ID NO:2. In some embodiments, the CRBN amino acid domain comprises at least about 91%, 92%, 93%, 94%, 95%, 96%, 97%, 98%, 99%, or 100% sequence identity to SEQ ID NO:2. In some aspects, the CRBN amino acid domain comprises at least about 85% sequence identity to SEQ ID: NO: 2.

In various aspects, the CRBN amino acid domain comprises at least about 70%, at least about 75%, at least about 80%, at least about 85%, or at least about 90% sequence identity to SEQ ID NO:3. In some embodiments, the CRBN amino acid domain comprises at least about 91%, 92%, 93%, 94%, 95%, 96%, 97%, 98%, 99%, or 100% sequence identity to SEQ ID NO:3. In some aspects, the CRBN amino acid domain comprises at least about 85% sequence identity to SEQ ID: NO: 3.

In various aspects, the CRBN amino acid domain comprises at least about 70%, at least about 75%, at least about 80%, at least about 85%, or at least about 90% sequence identity to SEQ ID NO:4. In some embodiments, the CRBN amino acid domain comprises at least about 91%, 92%, 93%, 94%, 95%, 96%, 97%, 98%, 99%, or 100% sequence identity to SEQ ID NO:4. In some aspects, the CRBN amino acid domain comprises at least about 85% sequence identity to SEQ ID: NO: 4. In various aspects, the CRBN amino acid domain comprises at least about 70%, at least about 75%, at least about 80%, at least about 85%, or at least about 90% sequence identity to SEQ ID NO: 5. In some embodiments, the CRBN amino acid domain comprises at least about 91%, 92%, 93%, 94%, 95%, 96%, 97%, 98%, 99%, or 100% sequence identity to SEQ ID NO:5. In some aspects, the CRBN amino acid domain comprises at least about 85% sequence identity to SEQ ID: NO: 5. In various aspects, the CRBN amino acid domain comprises at least about 70%, at least about 75%, at least about 80%, at least about 85%, or at least about 90% sequence identity to SEQ ID NO:6. In some embodiments, the CRBN amino acid domain comprises at least about 91%, 92%, 93%, 94%, 95%, 96%, 97%, 98%, 99%, or 100% sequence identity to SEQ ID NO:6.

In some aspects, the CRBN amino acid domain comprises at least about 85% sequence identity to SEQ ID: NO: 6. In various aspects, the CRBN amino acid domain comprises at least about 70%, at least about 75%, at least about 80%, at least about 85%, or at least about 90% sequence identity to SEQ ID NO:7. In some embodiments, the CRBN amino acid domain comprises at least about 80%, 85%, 90%, 91%, 92%, 93%, 94%, 95%, 96%, 97%, 98%, 99%, or 100% sequence identity to SEQ ID NO:7. In some aspects, the CRBN amino acid domain comprises at least about 85% sequence identity to SEQ ID: NO: 7. In some embodiments, the disclosure relates to a nucleic acid molecule comprising a nucleic acid sequence comprising at least about 80%, 85%, 90%, 91%, 92%, 93%, 94%, 95%, 96%, 97%, 98%, 99%, or 100% sequence identity to SEQ ID NO:8, or a functional fragment thereof. In some embodiments, the disclosure relates to a nucleic acid molecule comprising a nucleic acid sequence comprising at least about 80%, 85%, 90%, 91%, 92%, 93%, 94%, 95%, 96%, 97%, 98%, 99%, or 100% sequence identity to SEQ ID NO:9, or a functional fragment thereof. In some embodiments, the disclosure relates to a nucleic acid molecule comprising: (i) a nucleic acid sequence comprising at least about 80%, 85%, 90%, 91%, 92%, 93%, 94%, 95%, 96%, 97%, 98%, 99%, or 100% sequence identity to SEQ ID NO:8, or a functional fragment thereof; and (ii) a nucleic acid sequence comprising at least about 80%, 85%, 90%, 91%, 92%, 93%, 94%, 95%, 96%, 97%, 98%, 99%, or 100% sequence identity to SEQ ID NO:9, or a functional fragment thereof. In some embodiments, the nucleic acid molecule further comprises a nucleic acid sequence encoding an oncoprotein that associates or binds to a target protein.

In various aspects, the composition further comprises a third domain comprising a tag. In some embodiments, the tag is a fluorescent protein. In a further aspect, the tag is fluorescent protein. In further aspects, the tag is free of green fluorescent protein.

In various aspects, the composition further comprises an inducer. Examples of inducers include, but are not limited to, lenalidomide, pomalidomide, or a derivative thereof. Thus, in various aspects, the inducer is lenalidomide. In a further aspect, the inducer is pomalidomide.

In various aspects, the first domain and second domain are dimerized at a concentration, in respect to each domain, from about 100 nM to about 900 nM, about 100 nM to about 700 nM, about 100 nM to about 500 nM, about 100 nM to about 300 nM, about 300 nM to about 900 nM, about 500 nM to about 900 nM, about 700 nM to about 900 nM, about 200 nM to about 800 nM, about 300 nM to about 700 nM, or about 400 nM to about 600 nM. In a further aspect, the first domain and second domain are dimerized at a concentration from about 100 nM to about 900 nM.

In various aspects, the first or second amino acid sequence further comprises a targeting domain; or wherein the first or second amino acid sequence is conjugated to a targeting domain by a linker. In a further aspect, the targeting domain is an antibody or antibody fragment. In a still further aspect, the antibody or antibody fragment comprise a first complementarity region (CDR) that recognizes a portion of any epitope of the target protein.

In various aspects, the first domain and the second domain are dimerized by the presence of an inducer. In some embodiments, the inducer is chosen from: rapamycin, a cereblon E3Ligase modulator, lenalidomide or a derivative of any of the foregoing.

In various aspects, the condensing agent is lenalidomide or acceptable salts thereof.

In some embodiments, the first or second amino acid domain is fused to one or more oncoproteins (cancer antigens) that may associate with a second oncoprotein in a subject or in vitro. Formation of a granule in the presence of an inducer can trap the first or second oncoproteins in the granule that facilitates detection, quantification, isolation or even manipulation or sequencing of the oncoproteins. In some embodiments, the first domain further comprises an oncoprotein or functional fragment either fused on its amino or carboxy terminus. In some embodiments the first domain may separate the oncoprotein by an amino acid linker. In some embodiments, the first domain comprises a zinc finger amino acid sequence fused contiguously to an oncoprotein on either its carboxy or amino terminus. In some embodiments, the second domain further comprises an oncoprotein or functional fragment either fused on its amino or carboxy terminus. In some embodiments the second domain may separate the oncoprotein by an amino acid linker. In some embodiments, the second domain comprises a cereblon amino acid sequence fused contiguously to an oncoprotein on either its carboxy or amino terminus.

In various aspects, the first or second amino acid domain further comprise an amino acid sequence comprising one or a combination of: MYC, YAP, TAZ, YAP-MAML2, or a functional fragment thereof. Thus, in a further aspect, the first or second amino acid domain further comprise an amino acid sequence comprising MYC or a functional fragment thereof. In a still further aspect, the first or second amino acid domain further comprise an amino acid sequence comprising YAP or a functional fragment thereof. In yet a further aspect, the first or second amino acid domain further comprise an amino acid Sequence comprising TAZ or a functional fragment thereof. In an even further aspect, the first or second amino acid domain further comprise an amino acid sequence comprising YAP-MAML2 or a functional fragment thereof. In a still further aspect, the first or second amino acid domain further comprise an amino acid sequence comprising a combination of: MYC, YAP, TAZ, YAP-MAML2, or a functional fragment thereof. In yet a further aspect, the first or second amino acid domain further comprise an amino acid sequence comprising two of: MYC, YAP, TAZ, YAP-MAML2, or a functional fragment thereof. In an even further aspect, the first or second amino acid domain further comprise an amino acid sequence comprising three of: MYC, YAP, TAZ. YAP-MAML2, or a functional fragment thereof. In a still further aspect, the first or second amino acid domain further comprise an amino acid comprising each of: MYC, YAP, TAZ. YAP-MAML2, or a functional fragment thereof.

In various aspects, the composition is in the form of an amino acid granule that is free of a lipid membrane. In various aspects, the composition is in the form of an amino acid granule that is free of material in the liquid phase. In further aspects, the granule is from about 0.1 micron to about 30 microns in width, from about 0.1 micron to about 25 microns in width, from about 0.1 micron to about 20 microns in width, from about 0.1 micron to about 15 microns in width, from about 0.1 micron to about 10 microns in width, from about 0.1 micron to about 5 microns in width, from about 5 micron to about 30 microns in width, from about 10 microns to about 30 microns in width, from about 15 microns to about 30 microns in width, from about 20 microns to about 30 microns in width, from about 25 microns to about 30 microns in width, from about 5 microns to about 25 microns in width, or from about 10 microns to about 20 microns in width. In still further aspects, the granule is from about 0.1 micron to about 30 microns in width.

The disclosure relates to an amino acid sequence comprising a first domain and a second domain wherein the first or second domain further comprise a targeting domain fused to either end of the amino acid sequence that is a first or second amino acid domain. In some embodiments, the first or second domain comprise a targeting domain that comprises an amino acid sequence tha comprises at least about 70%, 75%, 80%, 85%, 90%, 91%, 92%, 93%, 94%, 95%, 96%, 97%, 98%, 99%, or about 100% identity to MYC, YAP, TAZ, or YAP-MAML2.

HumanMYC
(SEQ ID NO: 10)
mdffrvvenq qppatmplnv sftnmydld ydsvqpyfyc deeenfyqqq qqselqppap sediwkkfel lptpplspsr
rsglcspsyv avtpfslrgd ndggggsfst adqlemvtel lggdmvnqsf icdpddetfi kniiiqdcmw sgfsaaaklv
seklasyqaa rkdsgspnpa rghsvestss lylqdlsaaa secidpsvvf pyplndsssp kscasqdssa fspssdslls
stesspqgsp eplvlheetp pttssdseee qedeeeidvv svekrqapgk rsesgspsag ghskpphspl vlkrchvsth
qhnyaappst rkdypaakrv kldsvrvlrq isnnrkctsp rssdteenvk rrthnvlerq rmelkrsff alrdqipele
nnekapkvvi lkkatayils vqaeeqklis eedllrkrre qlkhkleqlr nsca
human YAP
(SEQ ID NO: 11)
mdpgqqpppq papqgqgqpp sqppqgqgpp sgpgqpapaa tqaapqappa ghqivhvrgd
setdlealfn avmnpktanv pqtvpmrlrk lpdsffkppe pkshsrqast dagtagaltp
qhvrahsspa slqlgavspg tltptgvvsg paatptaqhl rqssfeipdd vplpagwema
ktssgqryfl nhidqtttwq dprkamlsqm nvtaptsppv qqnmmnsasg plpdgweqam
tqdgeiyyin hknkttswld prldprfamn qrisqsapvk qppplapqsp qggvmggsns
nqqqqmrlqq lqmekerlrl kqqellrqam minpstans pkcqelalrs qlptleqdgg
tqnpvsspgm sqelrtmttn ssdpflnsgt yhsrdestds glsmssysvp rtpddflnsv
demdtgdtin qstlpsqqnr fpdyleaipg tnvdlgtleg dgmniegeel mpslqealss
dilndmesvl aatkldkesf ltwl
TAZ
(SEQ ID NO: 12)
mnpasapppl pppgqqvihv tqdldtdlea lfnsvmnpkp sswrkkilpe sffkepdsgs
hsrqsstdss gghpgprlag gaqhvrshss paslqlgtga gaagspaqqh ahlrqqsydv
tdelplppgw emtftatgqr yflnhiekit twqdprkamn qplnhmnlhp avsstpvpqr
smavsqpnlv mnhqhqqqma pstlsqqnhp tqnppaglms mpnalttqqq qqqklrlqri
qmererirmr qeelmrqeaa lcrqlpmeae tlapvqaavn pptmtpdmrs itnnssdpfl
nggpyhsreq stdsglglgc ysvpttpedf lsnvdemdtg enagqtpmni npqqtrfpdf
ldclpgtnvd lgtlesedli plfndvesal nksepfltwl
MAML2
(SEQ ID NO: 13)
mgdtappqap agglggasga gllgggsvtp rvhsaiverl rariaverqh hlscegryer
graessdrer estlqllslv qhgqgarkag khtkatataa tttapppppa appaasqaaa
taapppppdy hhhhqqhlln ssnnggsggi ngeqqppast pgdqmsali alqgslkrkq
vvnlspansk rpngfvdnsf ldikrirvge nlsagqgglq inngqsqims gtlpmsqapl
rktntlpsht hspgnglfnm glkevkkepg etlscskhmd gqmtqenifp nrygddpgeq
lmdpelqelf neltnisvpp msdlelenmi natikqddpf nidlgqqsqr stprpslpme
kivikseysp gltqgpsgsp qlrppsagpa fsmansalst sspipsvpqs qaqpqtgsga
sralpswqev shaqqlkqia anrqqharmq qhqqqhqptn wsalpssagp spgpfgqeki
pspsfgqqtf spqsspmpgv aggsgqskvm anymykagps aqgghldvlm qqkpqdlsrs
finnphpame prqgntkplf hfnsdqanqq mpsvlpsqnk psllhytqqq qqqqqqqqqq.
qqqqqpssqp aqslpsqpll rsplplqqkl llqqmqnqpi agmgyqvsqq qrqdqhsvvg
qntgpspspn pcsnpntgsg ymnsqqslln qqlmgkkqtl qrqimeqkqq lllqqqmlad
aekiapqdqi nrhlsrpppd ykdqrmvgn mqptaqysgg sstislnsnq alanpvstht
iltpnsslls tshgtrmpsl stavqnmgmy gnlpcnqpnt ysvtsgmnql tqqmpkqll
anqnnpmmpr pptlgpsnnn nvatfgagsv gnsqqlrpnl thsmasmppq rtsnvmitsn
ttapnwasqe gtskqqealt sagvrfptgt paaytpnqsl qqavgsqqfs qravappnql
tpavqmrpmn qmsqtingqt mgplrglnlr pnqlstqilp ninqsgtgln qsrtginqpp
sltpsnfpsp nqssrafqgt dhssdlafdflsqqndnmgp alnsdadfid sllktepgnd
dwmkdinlde ilgnns

In some embodiments, the CRBN amino acid domain or the zinc finger domain comprises at least about 70%, at least about 75%, at least about 80%, at least about 85%, or at least about 90% sequence identity to SEQ ID NO:10. In some embodiments, the CRBN amino acid domain or the zinc finger domain comprises at least about 91%, 92%, 93%, 94%, 95%, 96%, 97%, 98%, 99%, or 100% sequence identity to SEQ ID NO:10. In some aspects, the CRBN amino acid domain or the zinc finger domain comprises at least about 85% sequence identity to SEQ ID: NO: 10.

In some embodiments, the CRBN amino acid domain or the zinc finger domain comprises at least about 70%, at least about 75%, at least about 80%, at least about 85%, or at least about 90% sequence identity to SEQ ID NO:11. In some embodiments, the CRBN amino acid domain or the zinc finger domain comprises at least about 91%, 92%, 93%, 94%, 95%, 96%, 97%, 98%, 99%, or 100% sequence identity to SEQ ID NO: 11. In some aspects, the CRBN amino acid domain or the zinc finger domain comprises at least about 85% sequence identity to SEQ ID: NO: 11.

In some embodiments, the CRBN amino acid domain or the zinc finger domain comprises at least about 70%, at least about 75%, at least about 80%, at least about 85%, or at least about 90% sequence identity to SEQ ID NO:12. In some embodiments, the CRBN amino acid domain or the zinc finger domain comprises at least about 91%, 92%, 93%, 94%, 95%, 96%, 97%, 98%, 99%, or 100% sequence identity to SEQ ID NO: 12. In some aspects, the CRBN amino acid domain or the zinc finger domain comprises at least about 85% sequence identity to SEQ ID: NO: 12.

In some embodiments, the CRBN amino acid domain or the zinc finger domain comprises at least about 70%, at least about 75%, at least about 80%, at least about 85%, or at least about 90% sequence identity to SEQ ID NO: 13. In some embodiments, the CRBN amino acid domain or the zinc finger domain comprises at least about 91%, 92%, 93%, 94%, 95%, 96%, 97%, 98%, 99%, or 100% sequence identity to SEQ ID NO:13. In some aspects, the CRBN amino acid domain or the zinc finger domain comprises at least about 85% sequence identity to SEQ ID: NO: 13.

In some embodiments, the CRBN amino acid domain or the zinc finger domain comprises a functional fragment of any of SEQ ID: NO: 10, SEQ ID: NO: 11, SEQ ID: NO: 12 or SEQ ID: NO: 13 that is at least about 70%, at least about 75%, at least about 80%, at least about 85%, or at least about 90% sequence identity to any of SEQ ID: NO: 10, SEQ ID: NO: 11, SEQ ID: NO: 12 or SEQ ID: NO: 13. In some embodiments, the CRBN amino acid domain or the zinc finger domain comprises at least about 91%, 92%, 93%, 94%, 95%, 96%, 97%, 98%, or 99%, sequence identity to any of SEQ ID: NO: 10, SEQ ID: NO: 11, SEQ ID: NO: 12 or SEQ ID: NO: 13. In some aspects, the CRBN amino acid domain or the zinc finger domain comprises at least about 85% sequence identity to any of SEQ ID: NO: 10, SEQ ID: NO: 11, SEQ ID: NO: 12 or SEQ ID: NO: 13 any of SEQ ID: NO: 10, SEQ ID: NO: 11, SEQ ID: NO: 12 or SEQ ID: NO: 13.

In some embodiments, compositions and pharmaceutical compositions of the disclosure comprise a first amino acid domain, or a second amino acid domain, or first and second amino acid sequence domain, wherein the first domain comprises a zinc finger amino acid sequence or a functional fragment thereof, the second domain comprises a CRBN amino acid sequence or functional fragment thereof; and wherein the first or second domain further comprises a targeting domain, wherein the targeting domain comprises an amino acid sequence that an animal transcription factor, or functional fragment thereof. In some embodiments, compositions and pharmaceutical compositions of the disclosure comprise a first amino acid domain, or a second amino acid domain, or first and second amino acid sequence domain, wherein the first domain comprises a zinc finger amino acid sequence or a functional fragment thereof, the second domain comprises a CRBN amino acid sequence or functional fragment thereof; and wherein the first or second domain further comprises a targeting domain, wherein the targeting domain comprises an amino acid sequence that a human transcription factor, or functional fragment thereof. In some embodiments, the human transcription factor comprises a functional fragment comprising at least about 85% sequence identity to about 50 to about 900 amino acids of any of SEQ ID: NO: 10, SEQ ID: NO: 11, SEQ ID: NO: 12 or SEQ ID: NO: 13. In some embodiments, the human transcription factor comprises a functional fragment comprising at least about 85% sequence identity to about 100 to about 800 amino acids of any of SEQ ID: NO: 10, SEQ ID: NO: 11. SEQ ID: NO: 12 or SEQ ID: NO: 13. In some embodiments, the human transcription factor comprises a functional fragment comprising at least about 85% sequence identity to about 100 to about 700 amino acids of any of SEQ ID: NO: 10, SEQ ID: NO: 11. SEQ ID: NO: 12 or SEQ ID: NO: 13. In some embodiments, the human transcription factor comprises a functional fragment comprising at least about 85% sequence identity to about 100 to about 600 amino acids of any of SEQ ID: NO: 10, SEQ ID: NO: 11, SEQ ID: NO: 12 or SEQ ID: NO: 13. In some embodiments, the human transcription factor comprises a functional fragment comprising at least about 85% sequence identity to an amino acid sequence from about 10 to about 100 amino acids of any of SEQ ID: NO: 10, SEQ ID: NO: 11, SEQ ID: NO: 12 or SEQ ID: NO: 13. In some embodiments, the human transcription factor comprises a functional fragment comprising at least about 85% sequence identity to to an amino acid sequence from about 10 to about 80 amino acids of any of SEQ ID: NO: 10, SEQ ID: NO: 11, SEQ ID: NO: 12 or SEQ ID: NO: 13. In some embodiments, the human transcription factor comprises a functional fragment comprising at least about 85% sequence identity to an amino acid sequence from about 10 to about 50 amino acids of any of SEQ ID: NO: 10, SEQ ID: NO: 11, SEQ ID: NO: 12 or SEQ ID: NO: 13. In some embodiments, the human transcription factor comprises a functional fragment comprising at least about 85% sequence identity to to an amino acid sequence from about 10 to about 25 amino acids of any of SEQ ID: NO: 10, SEQ ID: NO: 11, SEQ ID: NO: 12 or SEQ ID: NO: 13. In some embodiments, any of the foregoing functional fragments retain the transcription factor binding capacity to a binding partner. In some embodiments and disclosed methods, such association of the targeting domain to its target results in association of the binding partner to the composition and stimulation or activation of the amino acid sequences to form granules (solid phase or semi-solid phase) in the presence of one or a plurality of inducers. In some embodiments and disclosed methods, such association of the targeting domain to its target results in association of the binding partner to the composition and disruption of the amino acid sequences to form granules (dissolution of the granule) in the presence of one or a plurality of inducers.

In some embodiments, the compositions or pharmaceutical compositions comprise functional fragments of SEQ ID NO: 1 and are free of a full-length SEQ ID NO:1. In some embodiments, the compositions or pharmaceutical compositions comprise functional fragments of SEQ ID NO:2 and are free of a full-length SEQ ID NO:2. In some embodiments, the compositions or pharmaceutical compositions comprise functional fragments of SEQ ID NO:3 and are free of a full-length SEQ ID NO:3. In some embodiments, the compositions or pharmaceutical compositions comprise functional fragments of SEQ ID NO:4 and are free of a full-length SEQ ID NO:4. In some embodiments, the compositions or pharmaceutical compositions comprise functional fragments of SEQ ID NO:5 and are free of a full-length SEQ ID NO:5. In some embodiments, the compositions or pharmaceutical compositions comprise functional fragments of SEQ ID NO:6 and are free of a full-length SEQ ID NO:6. In some embodiments, the compositions or pharmaceutical compositions comprise functional fragments of SEQ ID NO:7 and are free of a full-length SEQ ID NO:7. In some embodiments, the compositions or pharmaceutical compositions comprise functional fragments of SEQ ID NO:8 and are free of a full-length SEQ ID NO:8. In some embodiments, any of the foregoing functional fragments retain the ability of the first domain to bind to the second domain in the presence of an inducer.

In some embodiments, the compositions or pharmaceutical compositions comprise functional fragments of SEQ ID NO:10 and are free of a full-length SEQ ID NO:10. In some embodiments, the compositions or pharmaceutical compositions comprise functional fragments of SEQ ID NO: 11 and are free of a full-length SEQ ID NO:11. In some embodiments, the compositions or pharmaceutical compositions comprise functional fragments of SEQ ID NO:11 and are free of a full-length SEQ ID NO:11. In some embodiments, the compositions or pharmaceutical compositions comprise functional fragments of SEQ ID NO: 12 and are free of a full-length SEQ ID NO:12. In some embodiments, any of the foregoing functional fragments retain the transcription factor binding capacity to a binding partner. In some embodiments and disclosed methods, such association of the targeting domain to its target results in association of the binding partner to the composition and stimulation or activation of the amino acid sequences to form granules (solid phase or semi-solid phase) in the presence of one or a plurality of inducers. In some embodiments and disclosed methods, such association of the targeting domain to its target results in association of the binding partner to the composition and disruption of the amino acid sequences to form granules (dissolution of the granule) in the presence of one or a plurality of inducers.

Compositions of the disclosure relate to those compositions comprising a nucleic acid sequence comprising SEQ ID NO:8 or a functional fragment thereof.

Compositions of the disclosure relate to those compositions comprising a nucleic acid sequence comprising SEQ ID NO:9 or a functional fragment thereof.

Compositions of the disclosure relate to those compositions comprising a nucleic acid sequence comprising SEQ ID NO:8 or a functional fragment thereof, and SEQ ID NO:9 or a functional fragment thereof.

Compositions of the disclosure relate to those compositions comprising a nucleic acid sequence comprising a first functional fragment of SEQ ID NO:8, and a second functional fragment of SEQ ID NO:9, wherein each of the functional fragment independently can be from about 10 to about 100 nucleotides and encodes a zinc finger domain or CRBN domain, respectively, that retain the ability to bind to each other in the presence of an inducer. Compositions of the disclosure relate to those compositions comprising a nucleic acid sequence comprising a first functional fragment of SEQ ID NO:8, and a second functional fragment of SEQ ID NO:9, wherein each of the functional fragment independently are about 80%, 85%, 90%, 95%, or 100% sequence identity to a nucleotide sequence from about 10 to about 100 nucleotides, and such nucleotide sequences encode a zinc finger domain or CRBN domain, respectively, that retains the ability to bind to the other domain in the presence of an inducer.

In some embodiments, the first amino acid sequence or the second amino acid sequence comprise a signaling domain, such as SUMO or a functional fragment thereof.

(SEQ ID NO: 14)
MSDQEAKPST EDLGDKKEGE YIKLKVIGQD SSEIHFKVKM
TTHLKKLKES YCQRQGVPMN SLRFLFEGQR IADNHTPKEL
GMEEEDVIEV YQEQTGGHST

In some embodiments the signaling domain is an NLS or a functional fragment thereof. In some embodiments, the CRBN amino acid domain or the zinc finger domain comprises at least about 70%, at least about 75%, at least about 80%, at least about 85%, or at least about 90% sequence identity to SEQ ID NO:14. In some embodiments, the CRBN amino acid domain or the zinc finger domain comprises at least about 91%, 92%, 93%, 94%, 95%, 96%, 97%, 98%, 99%, or 100% sequence identity to SEQ ID NO:14. In some aspects, the CRBN amino acid domain or the zinc finger domain comprises a functional fragment of SUMO that comprises at least about 85% sequence identity to a functional fragment of SEQ ID: NO: 14 that is from about 10 to about 20 amino acids in length.

In some embodiments, the first or second amino acid domain comprises a FKBP family protein, such as FKBP12. FKBP12 was first described in 1989 by Harding et al. (1989) and Siekierka et al. (1989). With a molecular weight of 12 kDa, it is the smallest member of the FKBP family. It contains the PPiase core domain, which is found in many FKBPs. It occurs in most species and tissues and is essential for mammalian life. Knock-out of FKBP12 in mice produced an embryonic lethal phenotype due to severe heart defects attributed to interference with the ryanodine receptor (Shou et al., 1998). Furthermore, FKBP12 is linked to various diseases including Alzheimer's and Parkinson's disease, but its distinct role still needs to be elucidated. The first ligands described for FKBP12 are the natural products Rapamycin and FK506. Both compounds are potent immunosuppressants in complex with FKBPs (best described for FKBP12) and act via a gain-of-function mechanism. The FKBP12-FK506 complex binds calcineurin (Griffith et al., 1995), a key enzyme in T-cell activation (Rosen and Schreiber, 1992; Kissinger et al., 1995), while the FKBP12-Rapamycin complex binds to the FKBP Rapamycin binding (FRB) domain of the mammalian target of Rapamycin (mTOR) (Liang et al., 1999; Banaszynski et al., 2005), a kinase involved in cell growth and cell proliferation (Waickman and Powell, 2012). In some embodiments, the first or second domain further comprises a FKBP domain comprising of FKBPL; FKBP1A; FKBP1B; FKBP2; FKBP3; FKBP4; FKBP5; FKBP6; FKBP7; FKBP8; FKBP9; FKBP10; FKBP11; FKBP14; FKBP15 or a functional fragment of any of the aforementioned proteins.

FKBP12
(SEQ ID NO: 15)
MGVEKQVIRP GNGPKPAPGQ TVTVHCTGFG KDGDLSQKFW STKDEGQKPF
SFQIGKGAVI KGWDEGVIGM QIGEV ARLRC SSDYAYGAGG FPAWGIQPNS
VLDFEIEVLS VQ

In some embodiments, the composition comprises a first amino acid sequence and a second amino acid sequence, wherein the first amino acid sequence comprises a zinc finger domain and the second amino acid sequence comprises a CRBN domain; and wherein the zinc finger domain or the CRBN domain comprises an amino acid sequence that is an FKBP family member or a functional fragment thereof. In some embodiments the zinc finger domain or CRBN domain comprises at least about 70%, at least about 75%, at least about 80%, at least about 85%, or at least about 90% sequence identity to SEQ ID NO:15. In some embodiments, the CRBN amino acid domain or the zinc finger domain comprises at least about 91%, 92%, 93%, 94%, 95%, 96%, 97%, 98%, 99%, or 100% sequence identity to SEQ ID NO: 15. In some aspects, the CRBN amino acid domain or the zinc finger domain comprises a functional fragment of FKBP12 or another family member that comprises at least about 85%, 95% or 100% sequence identity to a functional fragment of SEQ ID: NO: 15 that is from about 10 to about 100 amino acids in length. In the aforementioned embodiments, the zinc finger domain or the CRBN is free of the FKBP family member or a functional fragment thereof but comprises mTOR or a functional fragment thereof, a known dimerization partner to FKBP family members. mTOR

(SEQ ID NO: 16)
MLGTGPAAAT TAATTSSNVS VLQQFASGLK SRNEETRAKA AKELQHYVTM
ELREMSQEES TRFYDQLNHH IFELVSSSDA NERKGGILAI ASLIGVEGGN
ATRIGRFANY LRNLLPSNDP VVMEMASKAI GRLAMAGDTF TAEYVEFEVK
RALEWLGADR NEGRRHAAVL VLRELAISVP TFFFQQVQPF FDNIFVAVWD
PKQAIREGAV AALRACLILT TQREPKEMQK PQWYRHTFEE AEKGFDETLA
KEKGMNRDDR IHGALLILNE LVRISSMEGE RLREEMEEIT QQQLVHDKYC
KDLMGFGTKP RHITPFTSFQ AVQPQQSNAL VGLLGYSSHQ GLMGFGTSPS
PAKSTLVESR CCRDLMEEKF DQVCQWVLKC RNSKNSLIQM TILNLLPRLA
AFRPSAFTDT QYLQDTMNHV LSCVKKEKER TAAFQALGLL SVAVRSEFKV
YLPRVLDIIR AALPPKDFAH KRQKAMQVDA TVFTCISMLA RAMGPGIQQD
IKELLEPMLA VGLSPALTAV LYDLSRQIPQ LKKDIQDGLL KMLSLVLMHK
PLRHPGMPKG LAHQLASPGL TTLPEASDVG SITLALRTLG SFEFEGHSLT
QFVRHCADHF LNSEHKEIRM EAARTCSRLL TPSIHLISGH AHVVSQTAVQ
VVADVLSKLL VVGITDPDPD IRYCVLASLD ERFDAHLAQA ENLQALFVAL
NDQVFEIREL AICTVGRLSS MNPAFVMPFL RKMLIQILTE LEHSGIGRIK
EQSARMLGHL VSNAPRLIRP YMEPILKALI LKLKDPDPDP NPGVINNVLA
TIGELAQVSG LEMRKWVDEL FIIIMDMLQD SSLLAKRQVAL WTLGQLVAS
TGYVVEPYRK YPTLLEVLLN FLKTEQNQGT RREAIRVLGL LGALDPYKHK
VNIGMIDQSR DASAVSLSES KSSQDSSDYS TSEMLVNMGN LPLDEFYPAV
SMVALMRIFR DQSLSHHHTM VVQAITFIFK SLGLKCVQFL PQVMPTFLNV
IRVCDGAIRE FLFQQLGMLV SFVKSHIRPY MDEIVTLMRE FWVMNTSIQS
TIILLIEQIV VALGGEFKLY LPQLIPHMLR VFMHDNSPGR IVSIKLLAAI
QLFGANLDDY LHLLLPPIVK LFDAPEAPLP SRKAALETVD RLTESLDFTD
YASRIIHPIV RTLDQSPELR STAMDTLSSL VFQLGKKYQI FIPMVNKVLV
RHRINHQRYD VLICRIVKGY TLADEEEDPL IYQHRMLRSG QGDALASGPV
ETGPMKKLHV STINLQKAWG AARRVSKDDW LEWLRRLSLE LLKDSSSPSL
RSCWALAQAY NPMARDLFNA AFVSCWSELN EDQQDELIRS IELALTSQDI
AEVTQTLLNL AEFMEHSDKG PLPLRDDNGI VLLGERAAKC RAYAKALHYK
ELEFQKGPTP AILESLISIN NKLQQPEAAA GVLEYAMKHF GELEIQATWY
EKLHEWEDAL VAYDKKMDTN KDDPELMLGR MRCLEALGEW GQLHQQCCEK
WTLVNDETQA KMARMAAAAA WGLGQWDSME EYTCMIPRDT HDGAFYRAVL
ALHQDLFSLA QQCIDKARDL LDAELTAMAG ESYSRAYGAM VSCHMLSELE
EVIQYKLVPE RREIIRQIWW ERLQGCQRIV EDWQKILMVR SLVVSPHEDM
RTWLKYASLC GKSGRLALAH KTLVLLLGVD PSRQLDHPLP TVHPQVTYAY
MKNMWKSARK IDAFQHMQHF VQTMQQQAQH AIATEDQQHK QELHKLMARC
FLKLGEWQLN LQGINESTIP KVLQYYSAAT EHDRSWYKAW HAWAVMNFEA
VLHYKHQNQA RDEKKKLRHA SGANITNATT AATTAATATT TASTEGSNSE
SEAESTENSP TPSPLQKKVT EDLSKTLLMY TVPAVQGFFR SISLSRGNNL
QDTLRVLTLW FDYGHWPDVN EALVEGVKAI QIDTWLQVIP QLIARIDTPR
PLVGRLIHQL LTDIGRYHPQ ALIYPLTVAS KSTTTARHNA ANKILKNMCE
HSNTLVQQAM MVSEELIRVA ILWHEMWHEG LEEASRLYFG ERNVKGMFEV
LEPLHAMMER GPQTLKETSF NQAYGRDLME AQEWCRKYMK SGNVKDLTQA
WDLYYHVFRR ISKQLPQLTS LELQYVSPKL LMCRDLELAV PGTYDPNQPI
IRIQSIAPSL QVITSKQRPR KLTLMGSNGH EFVFLLKGHE DLRQDERVMQ
LFGLVNTLLA NDPTSLRKNL SIQRYAVIPL STNSGLIGWV PHCDTLHALI
RDYREKKKIL LNIEHRIMLR MAPDYDHLTL MQKVEVFEHA VNNTAGDDLA
KLLWLKSPSS EVWFDRRTNY TRSLAVMSMV GYILGLGDRH PSNLMLDRLS
GKILHIDFGD CFEVAMTREK FPEKIPFRLT RMLTNAMEVT GLDGNYRITC
HTVMEVLREH KDSVMAVLEA FVYDPLLNWR LMDTNTKGNK RSRTRTDSYS
AGQSVEILDG VELGEPAHKK TGTTVPESIH SFIGDGLVKP EALNKKAIQI
INRVRDKLTG RDFSHDDTLD VPTQVELLIK QATSHENLCQ CYIGWCPFW

In some embodiments, the composition comprises a first amino acid sequence and a second amino acid sequence, wherein the first amino acid sequence comprises a zinc finger domain and the second amino acid sequence comprises a CRBN domain; and wherein the zinc finger domain or the CRBN domain comprises an amino acid sequence that is an FKBP family member or a functional fragment thereof and the other amino acid sequence comprises mTOR or a functional fragment thereof. In some embodiments the zinc finger domain or CRBN domain comprises at least about 70%, at least about 75%, at least about 80%, at least about 85%, or at least about 90% sequence identity to SEQ ID NO:16. In some embodiments, the CRBN amino acid domain or the zinc finger domain comprises at least about 91%, 92%, 93%, 94%, 95%, 96%, 97%, 98%, 99%, or 100% sequence identity to SEQ ID NO:16. In some aspects, the CRBN amino acid domain or the zinc finger domain comprises a functional fragment of mTOR that comprises at least about 85%, 95% or 100% sequence identity to a functional fragment of SEQ ID: NO: 16 that is from about 10 to about 1000 amino acids in length. In some aspects, a first amino acid domain comprises a functional fragment of mTOR that comprises at least about 85%, 95% or 100% sequence identity to a functional fragment of SEQ ID: NO: 16 that is from about 10 to about 1000 amino acids in length and the second amino acid domain comprises a functional fragment of FKBP family member that comprises at least about 85%, 95% or 100% sequence identity to a functional fragment of SEQ ID: NO: 15 that is from about 10 to about 100 amino acids in length. If such embodiments are used for formation or dissolution methods disclosed herein, the methods may comprise a step of exposing the compositions to rapamycin or a salt or derivative thereof as an inducer.

### A. CELLS

Also provided herein are host cells comprising any disclosed composition. Thus, in various aspects, disclosed are cells comprising a composition comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof. The disclosure provides for isolated host cells or cells that comprise a nucleotide sequence that encodes: (i) a zinc finger domain; or (ii) a CRBN domain; or (iii) a first nucleotide sequence that encodes a zinc finger domain and a second nucleotide sequence that encodes a CRBN domain. In some embodiments, the host cells or cell comprises (i), (ii), or (iii), wherein one of the domain further comprises a nucleotide sequence that encodes a targeting domain. In some embodiments, the cell comprises a first nucleotide molecule that encodes the first domain and a second nucleotide molecule that encodes the second domain; or the cell comprises a single nucleotide molecule encoding (i) and (ii), optionally one of the first or second nucleotide sequences also encoding a third domina, the targeting domain. In some embodiments, the cell comprises the following:
(I): a first amino acid comprising a [zinc finger domain] and a second amino acid comprising a [CRBN domain]
(II) an amino acid sequence encoding a [zinc finger domain]-[CRBN domain], wherein, in either of (I) or (II), the zinc finger domain or the CRBN domain further comprises a targeting domain. In any of the aforementioned embodiments, the targeting domain comprises a human transcription factor or a functional fragment thereof.

In various aspects, the concentration of inducer is from about 10 nM to about 10 μM, about 10 nM to about 5 μM, about 10 nM to about 1 μM, about 10 nM to about 0.5 UM, about 10 nM to about 0.1 μM, about 0.1 μM to about 10 UM, about 0.5 μM to about 10 μM, about 1 μM to about 10 μM, about 5 μM to about 10 UM, about 0.1 μM to about 5 UM, or about 0.5 μM to about 1 μMin cytosol. In a further aspect, the concentration of inducer is from about 10 nM to about 10 UM in cytosol.

In various aspects, the composition of the first amino acid sequence and the second amino acid sequence comprises a droplet comprising a surface tension of from about 0.001 nN/m to about 1 nN/m, 0.001 nN/m to about 0.1 nN/m, 0.001 nN/m to about 0.01 nN/m. 0.01 nN/m to about 1 nN/m. 0.1 nN/m to about 1 nN/m, or 0.01 nN/m to about 0.1 nN/m. In a further aspect, the composition of the first amino acid sequence and the second amino acid sequence comprises a droplet comprising a surface tension of from about 0.001 nN/m to about 1 nN/m.

In various aspects, the cell is a cancer cell or a transformed cell. Thus, in a further aspect, the cell is a cancer cell. In a still further aspect, the cell is a transformed cell.

In various aspects, the cell is a 293T cell or a neuroblastoma (NB) cell. Thus, in various aspects, the cell is a 293T cell. In a still further aspect, the cell is a NB cell.

### B

### Methods of Screening Activity

Also provided herein are methods of screening activity of plurality of compounds, the method comprising: (a) exposing a compound to a composition comprising a first amino acid sequence and a second amino acid sequence for a time period sufficient to induce disassociation or association of the first amino acid sequence from or to, respectively, the second amino acid sequence, wherein the first amino acid sequence comprises a zinc finger domain, or a functional fragment thereof; and wherein the second amino acid sequence comprises a cereblon (CRBN) amino acid domain, or a functional fragment thereof.

In various aspects, the zinc finger domain comprises IZF1 or a functional fragment thereof.

In various aspects, the CRBN amino acid domain comprises at least 55%, at least 60%, at least 65%, at least 70%, at least 75%, at least 80%, at least 85%, or at least 90% sequence identity to SEQ ID NO:2. In a further aspect, the CRBN amino acid domain comprises at least 75% sequence identity to SEQ ID NO:2.

In various aspects, the composition further comprises a third domain comprising a tag. In a further aspect, the tag is a fluorescent protein, such as EGFP. In a still further aspect, the tag is free of green fluorescent protein.

In various aspects, the method further comprises a step of exposing the first and second amino acid sequences to an inducer. Examples of inducers include, but are not limited to, lenalidomide, pomalidomide, or a derivative thereof. Thus, in various aspects, the inducer is lenalidomide. In a further aspect, the inducer is pomalidomide.

In various aspects, the method further comprises the step of exposing the first amino acid sequence to the second amino acid sequence to an inducer for a sufficient time period to induce association or dissociation of the first amino acid sequence to or from, respectively, the second amino acid sequence with each other, wherein such step is performed before step (a).

In various aspects, the method further comprises the step of measuring the amount of association or disassociation in the presence of one or a plurality of compounds as compared to the association or disassociation in the absence of one or a plurality of compounds; and characterizing the activity of the one or plurality of compounds based upon the magnitude or inversion capillary velocity of the granule formation. In some embodiments of the compositions and methods, the granule has an inversion capillary velocity from about 1 to about 4 seconds per micron (s/μm). In some embodiments, the granule forms or dissolves at an inversion capillary velocity of from about 1 to about 4 seconds per micron (s/μm). In some embodiments, the granule forms or dissolves at an inversion capillary velocity of from about 1.5 to about 3.5 seconds per micron (s/μm). In some embodiments, the granule forms or dissolves at an inversion capillary velocity of from about 2 to about 4 seconds per micron (s/μm). In some embodiments, the granule forms or dissolves at an inversion capillary velocity of from about 1.5 to about 3 seconds per micron (s/μm). In some embodiments, the granule forms or dissolves at an inversion capillary velocity of from about 2 to about 3 seconds per micron (s/μm). In some embodiments, the average inversion capillary velocity is from about 3 to about 3.5 s/micron. The disclosure relates to any composition disclosed herein in granule phase (in the presence of inducer or condensing agent), wherein the composition comprises a granule with an inversion capillary velcoty of from about 1 to about 4 seconds per micron (s/μm). In some embodiments, the granule forms or dissolves at an inversion capillary velocity of from about 1 to about 4 seconds per micron (s/μm). In some embodiments, the granule forms or dissolves at an inversion capillary velocity of from about 1.5 to about 3.5 seconds per micron (s/μm). In some embodiments, the granule forms or dissolves at an inversion capillary velocity of from about 2 to about 4 seconds per micron (s/μm). In some embodiments, the granule forms or dissolves at an inversion capillary velocity of from about 1.5 to about 3 seconds per micron (s/μm). In some embodiments, the granule forms or dissolves at an inversion capillary velocity of from about 2 to about 3 seconds per micron (s/μm). In some embodiments, the average inversion capillary velocity is from about 3 to about 3.5 s/micron.

In various aspects, the first domain and second domain are dimerized at a concentration from about 100 nM to about 900 nM, about 100 nM to about 700 nM, about 100 nM to about 500 nM, about 100 nM to about 300 nM, about 300 nM to about 900 nM, about 500 nM to about 900 nM, about 700 nM to about 900 nM, about 200 nM to about 800 nM, about 300 nM to about 700 nM, or about 400 nM to about 600 nM. In a further aspect, the first domain and second domain are dimerized at a concentration from about 100 nM to about 900 nM. In some embodiments, the first domain and the second domain dimerize or associate in the presence of an inducer at any of the above-identified concentrations. The disclosure relates to a composition comprising a granule with a first and second domain at the concentration listed above.

The disclosure relates to a method of screening for inhibitors of granule formation comprising exposing a composition comprising an amino acid sequence comprising a first zinc finger domain and a second CRN domain to an inducer in the presence of a inhibitor candidate, wherein the inhibitor candidate is identified as an inhibitor of granule formation if the formation of the granule is slowed or completely disrupted under conditions sufficient to form a granule and the inhibitor candidate is not an inhibitor if granule formation occurs, in either case as compared to the formation rate of the granule in the absence of the inhibitor candidate. The disclosure also relates to a method of screening for inhibitors of granule formation comprising exposing a granule comprising an amino acid sequence comprising a first zinc finger domain and a second CRN domain to an inhibitor candidate in the presence of an inducer that induces association or dimerization of the first and second domains, wherein the inhibitor candidate is identified as an inhibitor of granule formation if the granule dissolves or beings to transition to liquid phase under conditions sufficient to form a granule; and the inhibit or candidate is not an inhibitor of granule formation if in the presence of the inducer, the granule does not degrade or dissolve; in either case as compared to the physical state of the granule in the absence of the inhibitor candidate.

In some embodiments, the cell comprises one or a plurality of granules from about 200 to about 400 nm in width (at their maximum width point).

### C. METHODS OF INDUCING PHASE SEPARATION AND/OR ISOLATING A PROTEIN

Also provided herein are methods of inducing phase separation in a solution, the solution comprising a composition comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof; the method comprising: (a) exposing a compound to a composition comprising a first amino acid sequence and a second amino acid sequence for a time period sufficient to induce disassociation or association of the first amino acid sequence and the second amino acid sequence.

Also provided herein are methods of isolating a protein from a solution comprising: (a) exposing a compound to a composition comprising a first amino acid sequence and a second amino acid sequence for a time period sufficient to induce disassociation or association of the first amino acid sequence and the second amino acid sequence; wherein the solution comprises a composition comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof. Also provided herein are methods of isolating a protein from a solution comprising: (a) exposing a compound to any composition disclosed herein.

In various aspects, the step of exposing further comprises a step of associating or disassociating the first and second domains in the presence of an inducer. Examples of inducers include, but are not limited to, lenalidomide, pomalidomide, or a derivative thereof. Thus, in various aspects, the inducer is lenalidomide. In a further aspect, the inducer is pomalidomide. In some embodiments, the inducer induces dissolution of the of the first amino acid sequence and the second amino acid sequence disclosed herein. In some embodiments, the induce is exposed to the first and second amino acid domains at a concentration identified in the Examples or Figures, or a concentration that is about the value identified in the Examples or Figures.

Disclosed are methods of measuring inversion velocity within a tissue or isolated host cell comprising positioning one or a plurality of tissues in any of the compositions disclosed herein; contacting the one or plurality of tissues or isolated host cells to one or a plurality of inducers in the presence of an agent; quantifying the inversion velocity of granule formation in density after contacting the one or plurality of tissues or cells to one or a plurality of agents; and calculating the difference in the inversion velocity prior to and after the step of contacting the one or plurality of tissues to one or a plurality of agents as compared to the inversion velocity of granule formation in same type of isolated host cells or tissue in the absence of the agent.

Also disclosed are methods of measuring intracellular or extracellular quantities of cellular amino acids or nucleic acid sequences in a culture comprising culturing one or a plurality of tissues in any of the composition disclosed herein; forming one or a plurality of granules in the culture by exposing the culture to an inducer; identifying and measuring a quantity of precipitated amino acid in a granule across the one or a plurality of tissues.

Also disclosed are methods of real-time imaging of tissue comprising culturing tissue within any of the tissue culture systems disclosed herein; and exposing the tissue culture system to digital imaging.

Also disclosed are methods of making any of the tissue culture systems disclosed herein comprising forming an interior chamber within a solid substrate; affixing tissue or isolated cells from a subject to the solid substrate; exposing the tissue or cells to one or more nucleic acid molecules disclosed herein (such as an nucleic acid molecule that encodes the first and/or second amino acid domains disclosed herein) to the tissue or isolated host cells for a period of time sufficient and under conditions sufficient to transfect or transform the tissue or isolated host cells with the nucleic acid molecule; exposing the tissue or isolated host cells to one or more inducers in the presence or absence of a test compound. In some embodiments, the method comprises culturing the tissue in cell culture medium at about 37 degrees Celsius; placing a reservoir of cell medium in fluid connection with at least the first length of tubing; placing a pump in operable connection to the at first length of tubing; and, optionally sealing the tissue within the solid substrate, such that the tissue is positioned within an internal cavity of the solid substrate in fluid communication with the reservoir.

The disclosure also relates to a method of testing the efficacy of a test substance comprising: exposing a three dimensional tissue comprising a granule disclosed herein to the test substance, in which the three dimensional cell culture comprises tissue secured to a solid substrate and in a culture chamber; and determining the effect of the test substance by measuring or observing a change in the granule formation or dissolution by exposure the test substance.

Also disclosed arc methods of printing isolated host cells on any of the tissue culture systems described herein; and evaluating the cells wherein the evaluating comprises forming a granule disclosed herein and isolating or imaging the contents of the granule. In some embodiments, the step of isolating the granule comprising separating the granule from the cell prior to sequencing one or more amino acids or nucleic acids in the granule. In some embodiments, the step of imaging comprises conducting light microscopy onto the granule, fluorescent microscopy on the granule and/or taking digital pictures of the granule to monitor morphometric changes of the granule within the cell. In some embodiments, the step of isolating the granule comprises removing the granule from the isolated host cell, and isolating the nucleic acids from the granule by dissolving the granule in a tube. In some embodiments, the step further comprises exposing the isolated granule to an inducer of dissolution, such as rapamycin or alcohol (such as ethanol) to phase separation the nucleic acid from the amino acids. In some embodiments, the methods comprise isolating RNA from the granule and then sequencing or analyzing the RNA to develop an expression profile of the cell. In some embodiments, the step of analyzing is the step of reverse transcribing the RNA, performing sequencing and/or exposing the RNA to a microarray of complementary nucleic acid sequences. In some embodiments, the step of analyzing the nucleic acid isolated from the granule comprises isolating DNA from the granule and exposing the DNA to one or a plurality of polymerase chain reaction (PCR) steps.

In some embodiments, RNA or DNA isolated from the granule can be hybridized in situ by, first, taking an isolated host cell comprising a granule, fixing it to a slide and then exposing it to one or more probes complementary to a panel of target nucleic acid molecules. After a period of time or the probes to hybridize or associate with target molecules in the cell, imaging analysis can be performed to quantify the amount of target molecules within the granule or cell. As an non-limiting example, antibodies or functional fragments of antibodies can be exposed to a fixed slide of the granule or the culture itself and one may quantify the number of target molecules in the cell. If the presence or quantity of tumor antigens or aberrant chromosomal events can be quantified, a pathologist can conclude whether a subject from which the sample are obtained has a graded cancer or neoplasia.

Also disclosed arc methods of producing a tissue culture, in-vitro culture of cells to monitor, quantify or isolate a target amino acid and/or target nucleic acid molecules that are or become trapped within one or more granules. In some embodiments, the target amino acid is an amino acid comprising blood and lymphatic microvascular networks, endothelial cells, smooth muscle cells, immune cells, neural cells, cancer or neoplastic cells, the method comprising inducing formation or dissolution of a granule by exposing the cells to an inducer.

Also disclosed arc methods of manufacturing a tissue culture, in-vitro model of cell growth. In some embodiments, the tissue culture comprises one or a plurality of isolated host cells comprising one or a plurality of amino acid sequences disclosed herein. In some embodiments, the isolated host cell or cells are chosen from: epithelial cells, endothelial cells, smooth muscle cells, immune cells, neural cells, cancer cells of any organ, transformed cell lines of any organ, and combinations thereof. In some embodiments, the cancer cells are primary cancer cells from the sample of a patient. In some methods of the cancer cells are those cancer cells with a dysfunction in a nuclear protein pathway, such that transcription factors such as those binding partners of oncoproteins are aberrantly upregulated. In some embodiments, the methods disclosed herein comprise harvesting a sample from a subject and seeding the cancer to a solid substrate. In some embodiments, the methods further comprise exposing the cells to one or more nucleotide molecule (such as a plasmid) comprising an expressible nucleic acid sequence encoding the one or plurality of amino acid sequences disclosed herein for a period of time sufficient to transform or transfect the isolated host cells. In further embodiments, the methods further comprise exposing the cells to a physiologically effective amount of an inducer in order to trigger formation of one or a plurality of granules in the isolated host cell.

In some embodiments, the system comprises a closed system for fluid flow in a fluid circuit. In some closed system embodiments, the tissue culture system comprises a cell medium reservoir, a first portion of tubing, a second portion of tubing and a three-dimensional tissue sample comprising one or plurality of cancer cells. In such embodiments, the tissue culture system comprises a pump (i.e. a peristaltic pump), in operable connection with a first portion of tubing, said pump capable of generating fluid flow through the first portion of tubing into the culture. In some embodiments, the first portion of tubing is a portion of tubing leading from a culture reservoir into the culture and the second portion of tubing is at a position distal from the first portion and carries fluid flow out of the closed system or exiting the system The pump is operable on one of the two tubing portions thus creating a flow of tissue culture media through the closed system in which the cells are growing. In some embodiments, the second portion of tubing is in fluid communication with an inlet of the pump such that cell media can be circulated through a fluid circuit. The tissue culture system can also comprise a gas exchanger for introduction of gas such as carbon dioxide into the system and/or a heating element. In some embodiments, the tissue culture system is maintained at about 37 degrees Celsius an about 5% carbon dioxide.

In some embodiments, the disclosure relates to a method of translocating amino acids and/or nucleic acid from the nucleus of a cell to the cytosol fraction of a cell comprising exposing a cell to any of the disclosed compositions and an inducer for a period sufficient to induce phase separation; and allowing a time period sufficient for the granule to translocate from the nucleus to the cytosol. In such embodiments, the zinc finger domain or the CRBN domain comprise a nuclear localization sequence (NLS), that permits the amino acid sequence movement into the nucleus. In some embodiments, exposure of the NLS occurs without an inducer of condensation, such that the NLS is not sterically hindered and becomes biologically active, whereas in the same embodiments, formation of the granule results in steric hindrance of the NLS sequence and shunt the granule formed upon association to the other domain in the presence of an inducer of condensation.

Any of the above-identified methods of the disclosure may be performed in from about 1 to about 3 minutes, or from about 1 to about 14 mins, or from about 3 to about 5 minutes, or from about 1 to about 5 minutes after addition of the inducer.

### D. VACCINES

Also provided herein are vaccines comprising: a first amino acid sequence, a second amino acid sequence; and a third amino acid sequence or a nucleic acid sequence encoding a third amino acid sequence; wherein the first amino acid sequence comprises a zinc finger domain, or a functional fragment thereof; and wherein the second amino acid sequence comprises a cereblon (CRBN) amino acid domain, or a functional fragment thereof; wherein the third amino acid sequence comprises a tumor antigen.

In various aspects, the third amino acid or the nucleic acid encoding the third amino acid is fused to the carboxy or amino terminal end of the zinc finger domain or the CRBN domain.

Tumor-associated antigens (TAA) can be loosely categorized as oncofetal (typically only expressed in fetal tissues and in cancerous somatic cells), oncoviral (encoded by tumorigenic transforming viruses), overexpressed/accumulated (expressed by both normal and neoplastic tissue, with the level of expression highly elevated in neoplasia), cancer-testis (expressed only by cancer cells and adult reproductive tissues such as testis and placenta), lineage-restricted (expressed largely by a single cancer histotype), mutated (only expressed by cancer as a result of genetic mutation or alteration in transcription), post-translationally altered (tumor-associated alterations in glycosylation, etc.), or idiotypic (highly polymorphic genes where a tumor cell expresses a specific “clonotype”, i.e., as in B cell, T cell lymphoma/leukemia resulting from clonal aberrancies). Although they are preferentially expressed by tumor cells, TAAs are oftentimes found in normal tissues. However, their expression differs from that of normal tissues by their degree of expression in the tumor, alterations in their protein structure in comparison with their normal counterparts or by their aberrant subcellular localization within malignant or tumor cells.

Examples of oncofetal tumor associated antigens include Carcinoembryonic antigen (CEA), immature laminin receptor, and tumor-associated glycoprotein (TAG) 72. Examples of overexpressed/accumulated include BING-4, calcium-activated chloride channel (CLCA) 2, Cyclin A1, Cyclin B1, 9D7, epithelial cell adhesion molecule (Ep-Cam), EphA3, Her2/neu, telomerase, mesothelin, orphan tyrosine kinase receptor (RORI), stomach cancer-associated protein tyrosine phosphatase 1 (SAP-1), and survivin.

Examples of cancer-testis antigens include the b melanoma antigen (BAGE) family, cancer-associated gene (CAGE) family, G antigen (GAGE) family, melanoma antigen (MAGE) family, sarcoma antigen (SAGE) family and X antigen (XAGE) family, CT9, CTIO, NY-ESO-1, L antigen (LAGE) 1, Melanoma antigen preferentially expressed in tumors (PRAME), and synovial sarcoma X (SSX) 2. Examples of lineage restricted tumor antigens include melanoma antigen recognized by T cells-1/2 (Melan-A/MART-1/2), Gp100/pmell 7, tyrosine-related protein (TRP) 1 and 2, P. polypeptide, melanocortin 1 receptor (MC IR), and prostate-specific antigen. Examples of mutated tumor antigens include P-catenin, breast cancer antigen (BRCA) 1/2, cyclin-dependent kinase (CDK) 4, chronic myelogenous leukemia antigen (CML) 66, fibronectin, p53, Ras, and TGF-PRII. An example of a post-translationally altered tumor antigen is mucin (MUC) 1. Examples of idiotypic tumor antigens include immunoglobulin (Ig) and T cell receptor (TCR).

In some embodiments, the antigen associated with the disease or disorder is selected from the group consisting of CD19, CD20, CD22, hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acetylcholine receptor, HMW-MAA, IL-22R-alpha, IL-13R-alpha, kdr, kappa light chain, Lewis Y, MUC16 (CA-125), PSCA. NKG2D Ligands, oncofetal antigen, VEGF-R2, PSMA, estrogen receptor, progesterone receptor, ephrinB2, CD123, CS-1, c-Mct and/or biotinylated molecules, and/or molecules expressed by HIV, HCV, HBV or other pathogens.

Exemplary tumor antigens include at least the following: carcinoembryonic antigen (CEA) for bowel cancers; CA-125 for ovarian cancer; MUC1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myeloma and in some lymphomas; CAI9-9 for colorectal, bile duct, and pancreatic cancer; chromogranin A for lung and prostate cancer; TA90 for melanoma, soft tissue sarcomas, and breast, colon, and lung cancer. Examples of TAAs are known in the art, for example in N. Vigneron, “Human Tumor Antigens and Cancer Immunotherapy,” BioMed Research International, vol. 2015, Article ID 948501, 17 pages, 2015. doi: 10.1155/2015/948501; Ilyas et al., J Immunol. (2015) December 1; 195 (11): 5117-5122; Coulie et al., Nature Reviews Cancer (2014) volume 14, pages 135-146; Cheever et al., Clin Cancer Res. (2009) September 1; 15 (17): 5323-37, which are incorporated by reference herein in its entirety.

Examples of oncoviral TAAs include human papilloma virus (HPV) L1, E6 and E7, Epstein-Barr Virus (EBV) Epstein-Barr nuclear antigen (EBNA) 1 and 2, EBV viral capsid antigen (VCA) Igm or IgG, EBV early antigen (EA), latent membrane protein (LMP) 1 and 2, hepatitis B surface antigen (HBsAg), hepatitis B e antigen (HBeAg), hepatitis B core antigen (HBcAg), hepatitis Bx antigen (HBxAg), hepatitis C core antigen (HCV core Ag), Human T-Lymphotropic Virus Type 1 core antigen (HTLV-1 core antigen), HTLV-1 Tax antigen, HTLV-1 Group specific (Gag) antigens, HTLV-1 envelope (Env), HTLV-1 protease antigens (Pro), HTLV-1 Tof, HTLV-1 Rof, HTLV-1 polymerase (Pro) antigen, Human T-Lymphotropic Virus Type 2 core antigen (HTLV-2 core antigen), HTLV-2 Tax antigen, HTLV-2 Group specific (Gag) antigens, HTLV-2 envelope (Env), HTLV-2 protease antigens (Pro), HTLV-2 Tof, HTLV-2 Rof, HTLV-2 polymerase (Pro) antigen, latency-associated nuclear antigen (LANA), human herpesvirus-8 (HHV-8) K8.1, Merkel cell polyomavirus large T antigen (LTAg), and Merkel cell polyomavirus small T antigen (sTAg).

Elevated expression of certain types of glycolipids, for example gangliosides, is associated with the promotion of tumor survival in certain types of cancers. Examples of gangliosides include, for example, GM1b, GD1c, GM3, GM2, GM1a, GD1a, GT1a, GD3, GD2, GD1b, GT1b, GQ1b, GT3, GT2, GT1c, GQ1c, and GP1c. Examples of ganglioside derivatives include, for example, 9-O-Ac-GD3, 9-O-Ac-GD2, 5-N-de-GM3, N-glycolyl GM3, NeuGcGM3, and fucosyl-GM1. Exemplary gangliosides that are often present in higher levels in tumors, for example melanoma, small-cell lung cancer, sarcoma, and neuroblastoma, include GD3, GM2, and GD2.

### E. METHODS OF MUTATING AN ENDOGENOUS DNA SEQUENCE

Also provided herein are methods of mutating an endogenous DNA sequence in a subject comprising exposing a cell of a subject to a disclosed composition. Thus, in various aspects, provided herein are methods of mutating an endogenous DNA sequence in a subject comprising exposing a cell of a subject to a composition comprising: a first amino acid sequence, a second amino acid sequence; and a third amino acid sequence or a nucleic acid sequence encoding a third amino acid sequence; wherein the first amino acid sequence comprises a zinc finger domain, or a functional fragment thereof; and wherein the second amino acid sequence comprises a cereblon (CRBN) amino acid domain, or a functional fragment thereof at a molecular ratio from about 1 to about 1; and wherein the third amino acid sequence or nucleic acid sequence encoding the third amino acid sequence is encapsulated within a particle comprising the first and second amino acid sequence; and wherein nucleic acid sequence encodes an enzyme or wherein the nucleic acid sequence is an sgRNA.

### F

### Methods of Forming a Particle

Also provided herein are methods of forming a particle in vivo or in vitro comprising: (a) exposing a compound to a composition comprising a first amino acid sequence and a second amino acid sequence for a time period sufficient to induce disassociation or association of the first amino acid sequence and the second amino acid sequence; wherein the solution comprising a composition comprising: a first amino acid sequence and a second amino acid sequence, wherein the first amino acid sequence comprises a zinc finger domain, or a functional fragment thereof; and wherein the second amino acid sequence comprises a cereblon (CRBN) amino acid domain, or a functional fragment thereof.

In various aspects, the step of exposing a compound to the composition comprises an inverse fusion velocity of form about 1 to about 10.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the disclosure concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the disclosure to the form disclosed herein. Also, it is intended to the appended claims be construed to include alternative embodiments.

All publications and patent applications cited in this specification are herein incorporated by reference in their entireties, and for any and all purposes, as if each individual publication or patent application were specifically and individually indicated to be incorporated by reference. In the event of an inconsistency between the present disclosure and any publications or patent application incorporated herein by reference in their entireties, the present disclosure controls.

### G. EXAMPLES

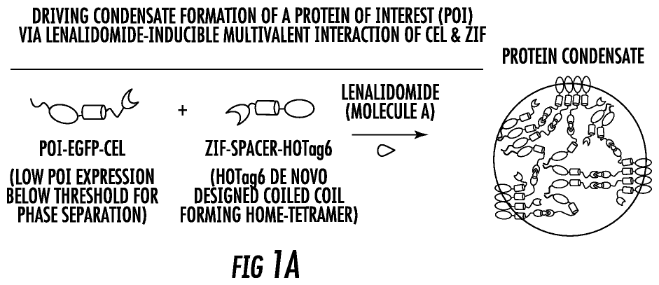

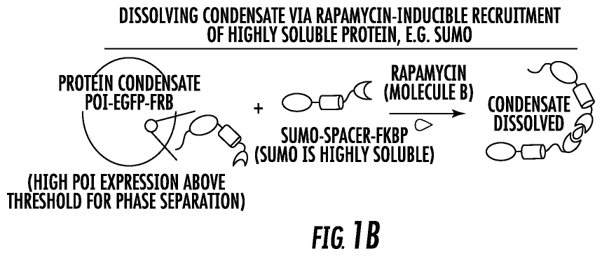

To investigate functional roles of protein phase separation without changing the protein expression level, two types of chemogenetic tools were designed that can: (1) use molecule A to drive protein phase separation, forming condensates (FIG. 1A); and (2) use molecule B to dissolve protein condensates (FIG. 1B). Specifically, multivalent interactions (inducible by molecule A) are used to dial up and drive protein phase separation, while in the other direction, highly soluble proteins are recruited by molecule B to dial down and dissolve protein condensates. As detailed herein, these chemogenetic tools have unique properties not available in existing tools, including: (1) dissolving protein condensates without changing expression level, which is powerful in understanding functional roles of condensates in living cells; (2) controlling protein phase separation with graded tuning by varying the dosage of the small molecules; (3) enabling duplex-control of phase separation by two proteins simultaneously with two orthogonal small molecule-induced systems. Overall, the phase-dial described herein will directly address one of the hardest and least addressed questions in biological phase-separation; namely, the physiological significance of phase-separation mechanisms. Additionally, the nature of the proposed tools will allow researchers to ask if conferring phase-separation on proteins not known to phase-separate in a test tube can change the differentiation pathway of a cell. This latter possibility has the potential to provide new ways to alter cell fate.

Representative examples of the disclosed compositions are illustrated in the following non-limiting methods, schemes, and examples.

### 1. Spark-on: Engineering Chemogenetic Tools for Dissecting Assembly Mechanisms and Functional Roles of Cytoplasmic and Nuclear Condensates

a. Structure-Based Design of Imid-Inducible Protein Heterodimer for Controlling PPI

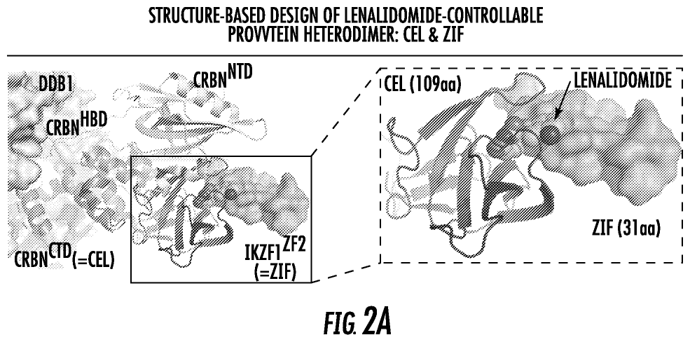

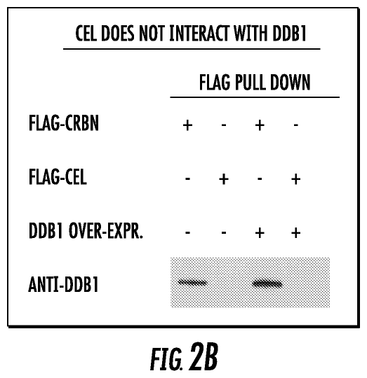

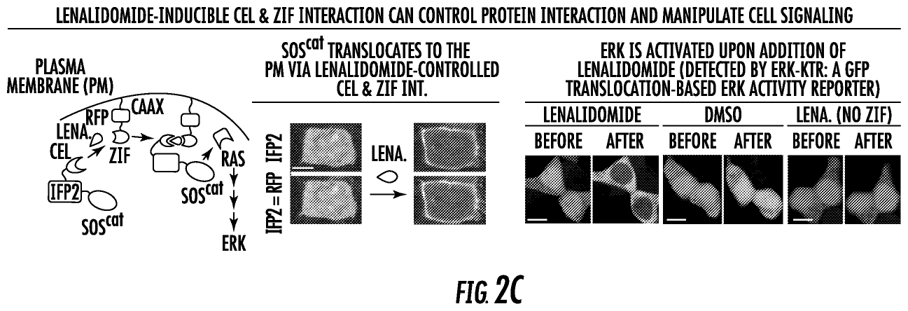

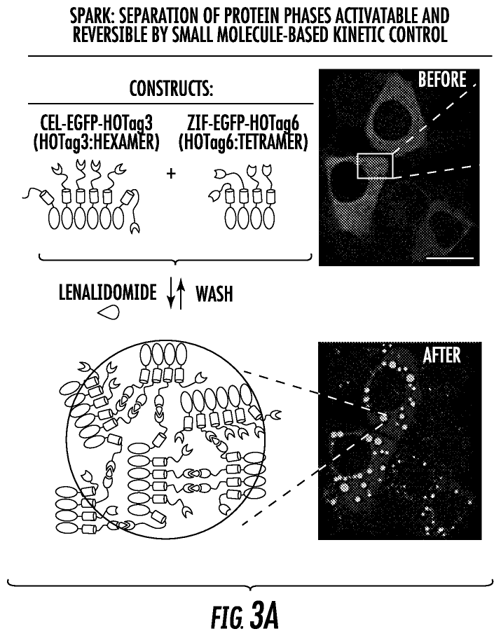

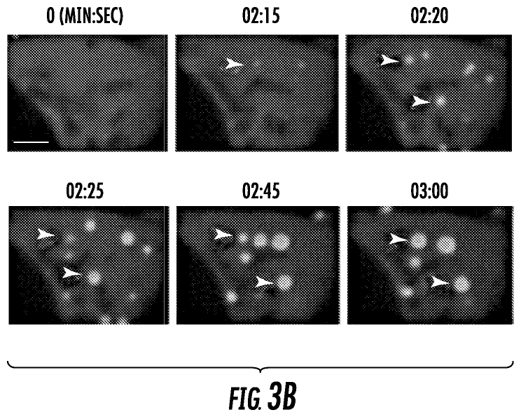

To design a small molecule inducible protein heterodimer with relatively weak interactions, we decided to engineer IMiDs-inducible PPI pair of cereblon (CRBN) and Ikaros (IKZF1) (Lu et al. (2014) Science 343:305-309). of this PPI pair is-0.1 μM (Petzold et al. (2016) Nature 532:127-130), which is-10-fold weaker than the rapamycin-inducible FKBP and Frb PPI pair (Banaszynski et al. (2005) J Am Chem Soc 127:4715-4721). IMiDs include thalidomide and lenalidomide, which are FDA-approved drugs against multiple myeloma and have no or little toxicity in other cells (Siu et al. (2017) Leukemia 31:1760-1769). IMID-dependent interactions between CRBN and IKZF1 bring the transcription factor IKZF1 to the cullin ring E3 ubiquitin ligase complex CUL4-RBX1-DDB1-CRBN via interaction between CRBN and the adaptor protein DDB1, resulting in ubiquitination and degradation of IKZF1. To design a stable IMID-controllable protein heterodimer, disruption of the interaction between CRBN and DDB1 was attempted. Structural studies of DDB1-CRBN-IKZF1 show that the helical bundle domain (HBD) of CRBN interacts with DDB1 but it does not bind lenalidomide or interact with IKZF1 (FIG. 2A) (Matyskiela et al. (2016) Nature 535:252-257). On the other hand, the C-terminal domain (CTD) of CRBN binds lenalidomide and interacts with IKZF1, without direct contact with DDB1. Additionally, the N-terminal domain (NTD) of CRBN does not appear to interact directly with DDB1 or IKZF1. Therefore, the NTD and HBD of CRBN were truncated initially, whereas the 109 amino acid (aa) CBRN CTD, which was renamed as CEL, was retained. Next, IKZF1 was truncated so that only the zinc finger 2 (ZF2) domain was retained, because ZF2 binds lenalidomide and interacts with CEL (FIG. 2A) (Matyskiela et al. (2016) Nature 535:252-257). The ZF2 of IKZF1 contains 31aa, and is referred to as ZIF (FIG. 2A). It was verified that CEL does not interact with endogenous DDB1 (or exogenous DDB1) based on immunoprecipitation, whereas CRBN interacts with DDB1 (FIG. 2B).

To demonstrate that the engineered CEL and ZIF can be used to control PPI via lenalidomide, whether this system can drive subcellular translocation of the GTP exchange factor SOS to the plasma membrane (PM) was tested. The presence of SOS in the PM can activate Ras and promote ERK activity via the MAPK pathway (FIG. 2C, left). For targeting SOS to the PM, ZIF was first fused to the CAAX motif tagged with a red fluorescent protein (RFP). Next, CEL was fused to the catalytic domain of SOS (referred to as SOScat), which was tagged with a near infrared fluorescent protein IFP2 that helps visualizing SOScat translocation (FIG. 2C, left). Upon addition of lenalidomide, SOScat translocated from the cytoplasm to the PM (FIG. 2C, middle) within 3-5 minutes. SOScat continued to accumulate at PM at later time points and membrane ruffling became visible. To determine whether SOScat translocation activates ERK, a GFP translocation-based ERK activity reporter called ERK-KTR was used, which translocates from the nucleus to the cytoplasm upon activation of endogenous ERK (Regot et al. (2014) Cell 157:1724-1734). Upon addition of lenalidomide, ERK-KTR translocated from the nucleus to the cytoplasm (FIG. 2C, right). In contrast, DMSO did not induce any ERK-KTR translocation, and lenalidomide alone did not induce ERK-KTR translocation in cells expressing the CEL-IFP2-SOScat without ZIF. ERK activation upon lenalidomide-induced SOScat translocation was further verified using another ERK activity reporter ERK-SPARK (Supporting FIG. S1) (Zhang et al. (2018) Mol Cell 69:334-345.e5). Thus, it was demonstrated that the lenalidomide-inducible CEL and ZIF heterodimer can be used to control PPI and cell signaling.

b. Multivalent PPI-Based Chemogenetic Tools for Controlling Protein Phase Separation

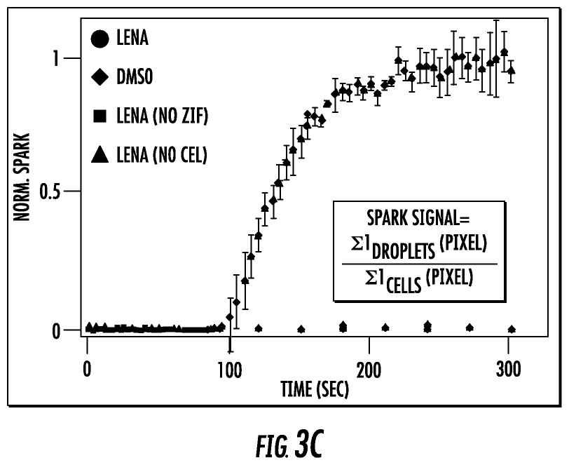

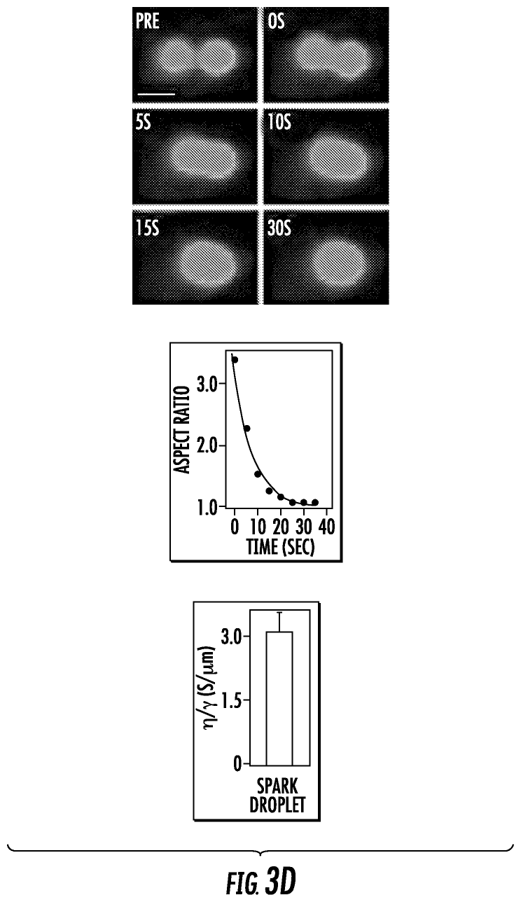

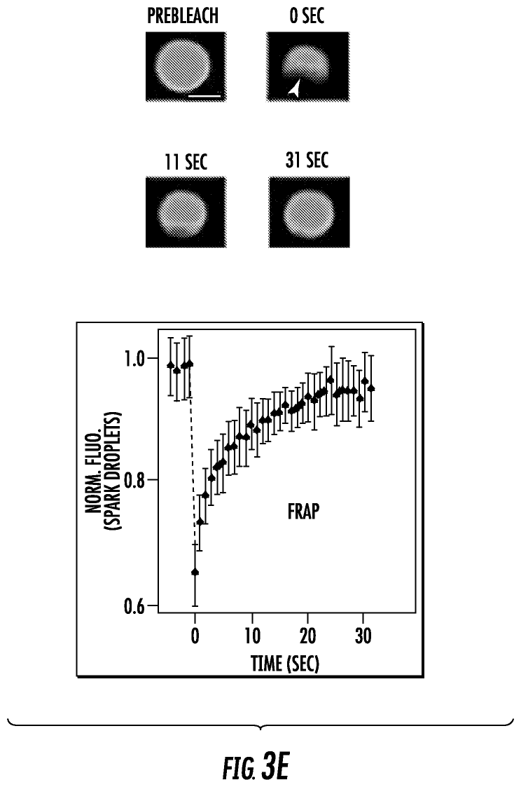

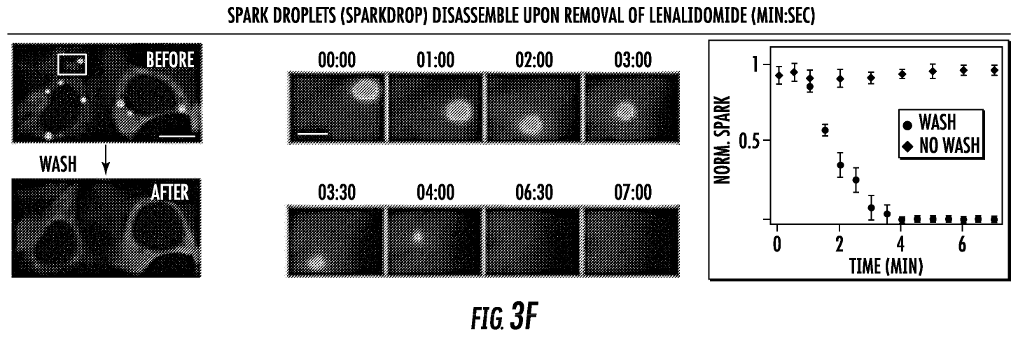

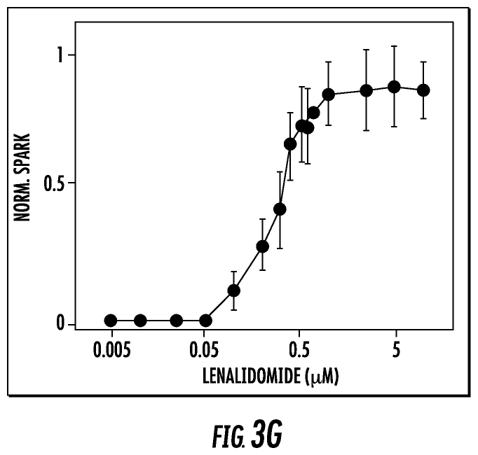

To engineer the CEL/ZIF system into a chemogenetic tool that can control protein LLPS, multivalency was introduced into the lenalidomide-dependent PPI system because multivalent PPI can drive protein phase separation (Li et al. (2012) Nature 483:336-340). CEL and ZIF were fused to the homo-oligomeric tags (HOTag) that are de novo designed coiled coils: CEL to HOTag3 (30 aa) and ZIF to HOTag6 (33 aa) (FIG. 3A, Supporting FIG. S2). HOTag3 and HOTag6 have previously been characterized as hexamer and tetramer, respectively (Zhang et al. (2018) Mol Cell 60:334-345.e5). To visualize phase separation, both constructs were tagged with the enhanced GFP (EGFP) (FIG. 3A), time-lapse imaging was used to visualize phase separation, growth and fusion of protein droplets (FIG. 3B-D). Addition of lenalidomide induced EGFP phase separation and formed brightly fluorescent droplets, suggesting that lenalidomide-dependent and HOTag-based multivalent PPI between CEL and ZIF leads to protein phase separation (FIG. 3A). At first, small protein droplets formed (−200-400 nm in diameter at 2 min: 15 sec after addition of lenalidomide), which rapidly grew into medium-size droplets, and these droplets continued to grow into relatively large droplets (−1.5 μm at 3 min) (FIG. 3B). This technology was named SPARK (separation of protein phases Activatable and Reversible by small molecule-based Kinetic control), or SPARK-ON. The time-lapse images were quantified by calculating SPARK signal, which is defined as the ratio of summarized pixel intensity of droplets divided by summary of pixel intensity of total cellular fluorescence. Quantitative analysis of the time-lapse fluorescence images revealed fast kinetics of lenalidomide-induced SPARK droplet formation (FIG. 3C). Control experiments showed that DMSO did not induce condensate formation, and that lenalidomide alone (lack of ZIF or CEL) could not induce protein phase separation (FIG. 3C).

Next, it was determined that these droplets have liquid-like properties based on two biophysical characterizations. First, time-lapse imaging was conducted and fusion events between droplets were characterized. Droplets fused and coalesced within a few seconds without changing total volume: two droplets with 2.4 μmin diameter fused into one droplet with 3 μmin diameter (FIG. 3D). The fusing droplets initially formed a dumbbell shape, which over time relaxed to a spherical shape (FIG. 3D, left panels). Quantitative analysis of the two fusing droplets showed that over time, the aspect ratio fitted a single exponential curve (FIG. 3D, middle panel), which is an established property of coalescing liquid droplets (Perie et al. (2016) Cell 165:1686-1697; Brangwynne et al. (2011) Proceedings of the National Academy of Sciences 108:4334-4339). Seven fusion events were characterized and the averaged inverse capillary velocity (=ri/y; here y is surface tension of the droplet; TJ is viscosity) to be 3.07±0.46 (s/μm) (FIG. 2C, right panel). Thus, quantitative analysis of the fusion events indicates that these micrometer-sized structures are liquid droplets. The second approach used is fluorescence recovery after photobleaching (FRAP), which showed strong recovery of fluorescence signal (>90% recovery) (FIG. 3D), suggesting that these condensates are highly dynamic, consistent with liquid-like properties. Therefore, the data including both the fusion events and FRAP, indicate that the lenalidomide-induced SPARK droplets are primarily liquid-like condensates. The SPARK droplet is here also referred to as SparkDrop.

It was further determined that upon removal of lenalidomide, the droplets disassembled within 5 minutes (FIG. 3E). The disassembly process was quantified by calculating SPARK signal. The reversibility of these SPARK droplets is consistent with the above characterization that they are liquid droplets. Solid-like condensates are known to be irreversible. Lastly, titration of lenalidomide in the cells showed that the degree of droplet formation (measured by SPARK signal) was dependent on the concentration of lenalidomide with half-to-maximum value—0.3 μM (FIG. 3F).

c. Phase Separation of Scaffold Protein G3BP1 Recruits Clients but not Vice Versa

Next, SparkDrop was applied to manipulate biomolecular condensates in the cytoplasm for understanding their assembly. To elucidate assembly process of condensate formation, composition of biomolecular condensates has been proposed to contain two types of macromolecules: scaffolds and clients (Banani et al. (2017) Nat Rev Mol Cell Biol, 1-14; Ditlev et al., J Mol Biol, in press; Banani et al. (2016) Cell 166:651-663). The scaffold proteins often contain a domain with high number of interaction valences, such as an IDR, which is largely responsible for driving phase separation (Wheeler and Hyman (2018) Philosophical Transactions of the Royal Society B: Biological Sciences 373:20170193-9). A leading model of condensate assembly is that condensates form by phase separation of scaffold proteins, which subsequently recruits clients that contain low number of interaction valence (Ditlev et al. J Mol Biol, in press). While this scaffold-client model might be simplified, it greatly helps understanding condensate assembly (Lyon et al. (2021) Nat Rev Mol Cell Biol, 1-21; Alberti et al. (2021) Nat Rev Mol Cell Biol, 1-18).

To examine this scaffold-client model and improve understanding of condensate assembly and composition, whether phase separation of a scaffold protein itself (without biological stimuli for condensate formation) would recruit client proteins, and whether phase separation of a client protein would recruit scaffold proteins, was tested. SparkDrop technology was applied to proteins of stress granules (SG). Recent studies have revealed that G3BP1 plays a central role in SG assembly (Sanders et al. (2020) Cell 181:306-324.e28; Yang et al. (2020) Cell 181:325-345.e28; Guillen-Boixet et al. (2020) Cell 181:346-361.el 7), and G3BP1 has been described as a scaffold protein for SG formation (Ditlev et al. J Mol Biol, in press; Kedersga et al. (2016) J Cell Biol 212:845-860). The client proteins of SG include the RNA-binding proteins FUS and TIA-1 (Ditlev et al. J Mol Biol, in press).

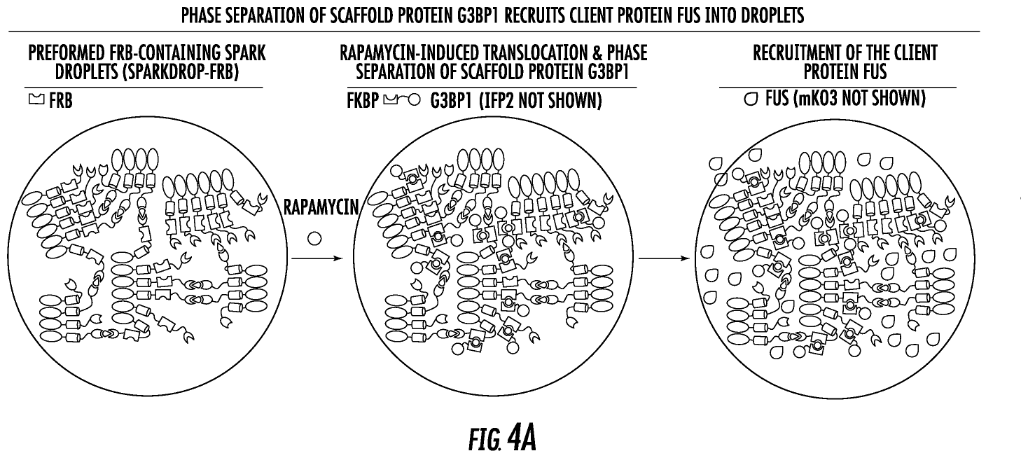

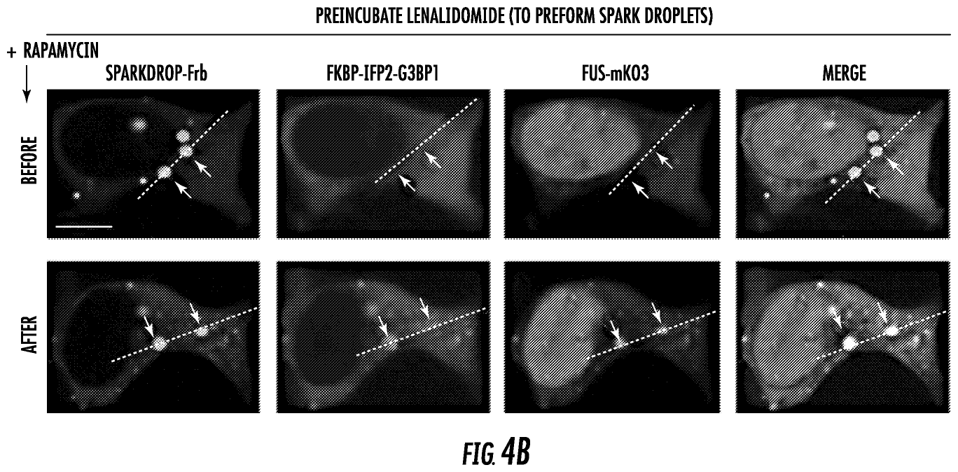

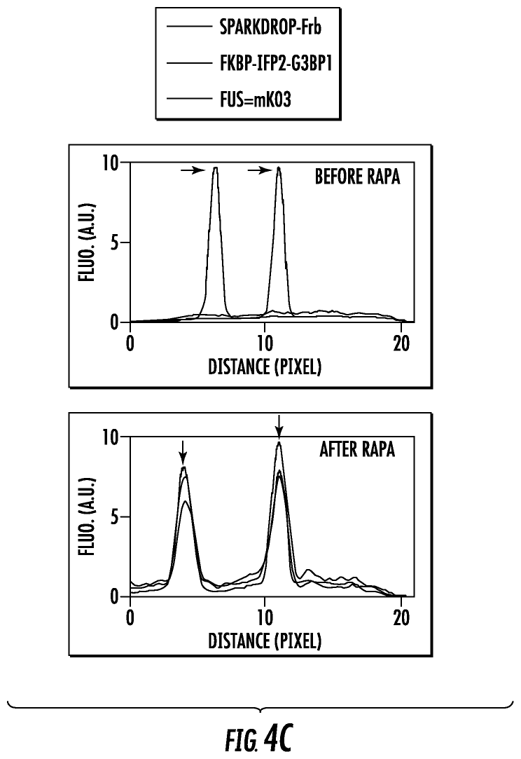

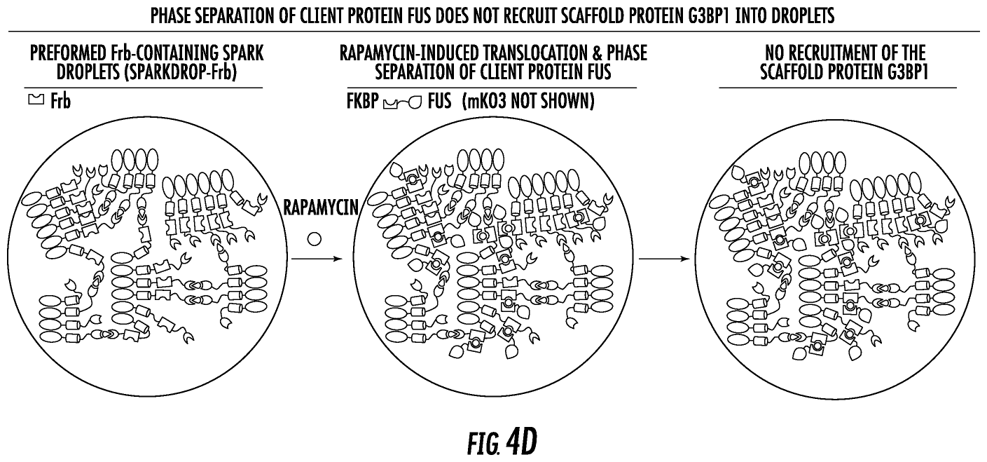

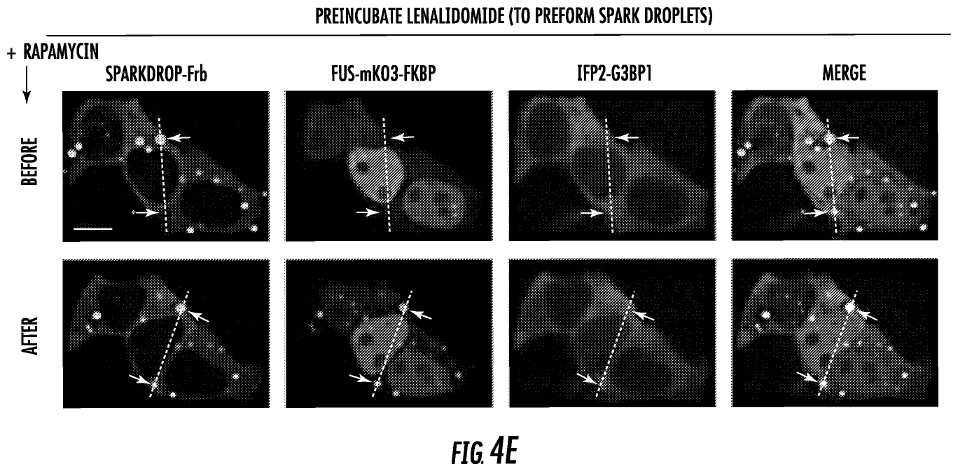

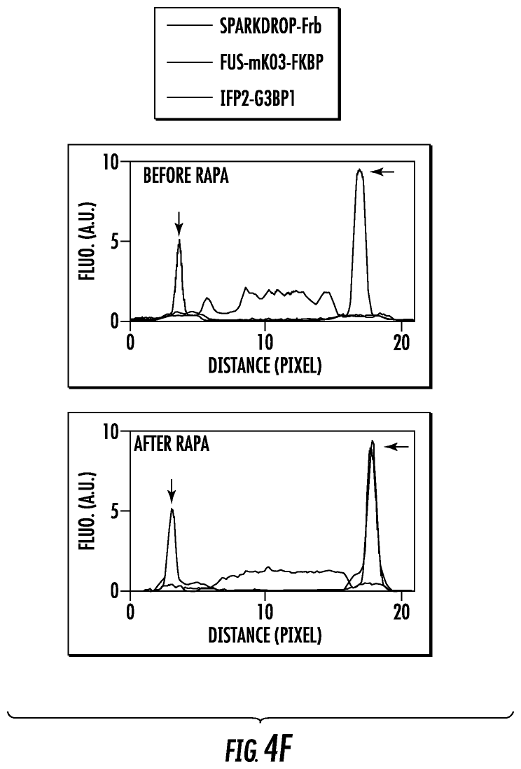

First, whether SparkDrop-induced G3BP1 condensates could recruit FUS and TIA-1 in living cells (without applying stress stimulus such as arsenite) was determed, and then whether FUS or TIA-1 phase separation could recruit G3BP1 was examined. Lenalidomide-inducible SparkDrop was first combined with the rapamycin-inducible FKBP and Frb heterodimer. Frb was incorporated into SparkDrop (referred to as SparkDrop-Frb, table S1), full-length G3BP1 was fused to FKBP tagged with IFP2, and FUS was tagged with a red fluorescent protein mKO3 (FIG. 4A). Droplets of SparkDrop-Frb were pre-formed by incubating the transfected cells with lenalidomide (FIG. 4B). Then, rapamycin was added to induce FKBP and Frb interaction, which should drive G3BP1 fusion protein into pre-formed droplets, resulting in formation of near-infrared fluorescent droplets. As shown in FIG. 4B and FIG. 4C, near-infrared G3BP1 droplets were observed-2 minutes after addition of rapamycin. Furthermore, red fluorescent FUS droplets were observed after the formation of G3BP1 droplets, which co-localized with both near-infrared and green droplets (FIG. 4B and FIG. 4C). In control experiments, rapamycin alone (lack of FKBP or Frb) did not induce G3BP1 phase separation or subsequent FUS recruitment (Supporting FIG. S3—data not shown). Thus, these imaging studies indicate that phase separation of the SG scaffold protein G3BP1 recruits the SG client protein FUS.

To induce phase separation of the client protein FUS and determine whether G3BP1 is recruited, FKBP was fused to FUS tagged with mKO3, and G3BP1 was fused to IFP2 (FIG. 4D). Droplets of SparkDrop-Frb were pre-formed by incubating the transfected cells with lenalidomide, expecting that subsequent addition of rapamycin should induce FKBP and Frb interaction and drive FUS fusion protein into pre-formed droplets, which based on the scaffold-client model, will unlikely recruit the scaffold protein G3BP1 (FIG. 4D). Time-lapse imaging revealed that rapamycin induced red fluorescent FUS droplets that co-localized with the pre-formed green droplets (FIG. 4E and FIG. 4F). However, no near-infrared fluorescent droplets were observed, indicating that G3BP1 was not recruited into the FUS droplets. In control experiments, rapamycin alone (lack of FKBP or Frb) did not induce FUS phase separation (Supporting FIG. S4). Thus, the imaging studies show that phase separation of the SG client protein FUS does not recruit the SG scaffold protein G3BP1.

Similar experiments were also conducted for another SG client protein, TIA-1, which showed that SparkDrop-induced G3BP1 condensates recruit TIA-1 (Supporting FIG. S5A-C—data not shown), and rapamycin alone (without FKBP or Frb) does not recruit TIA-1 (Supporting FIG. S6—data not shown). On the other hand, SparkDrop-induced TIA-1 condensates do not recruit G3BP1 (Supporting FIG. S5D-E—data not shown), and rapamycin alone (lack of FKBP or Frb) did not induce TIA-1 phase separation upon (Supporting FIG. S7—data not shown). Taken together, these data indicate that SparkDrop-induced phase separation of the SG scaffold protein G3BP1 can recruit the SG client proteins FUS and TIA-1, but not vice versa. Recently, many SG proteins that interact with G3BP1 have been identified (Jain et al. (2016) Cell 164:487-498), and the SparkDrop-based tools will be useful to further characterize these proteins for mechanistic understanding of SG formation and composition.

d. Sparkdrop Technology can Control YAP Condensate Formation

To demonstrate whether the SparkDrop technology can be used to induce nuclear condensates and to dissect their functional roles in gene regulation, the technology was applied to control YAP condensate formation in living cells. YAP is a transcriptional co-activator in the Hippo pathway, which is a highly conserved signaling pathway from Drosophila to mammals (Moya et al. (2018) Nat Rev Mol Cell Biol 96:1; Yu et al. (2015) Cell 163:811-828). YAP shuttles between the cytoplasm and the nucleus in response to diverse intracellular and extracellular cues including cell-cell contact and hyperosmolarity (Pocaterra et al. (2020) Journal of Cell Science 133: jcs230425-9). Upon activation, YAP translocates to the nucleus and forms condensates, which regulate gene transcription by interacting with the DNA-binding TEAD family transcriptional factors (Lin et al. (2017) Trends Biochem Sci 42:862-872; Cai et al. (2019) Nat Cell Biol, 1-25). YAP is thus a key effector in the Hippo pathway and plays a critical role in animal development and tissue homeostasis ((Moya et al. (2018) Nat Rev Mol Cell Biol 96:1; Yu et al. (2015) Cell 163:811-828; Manning et al. (2020) Development 147: devl 79069-10). Dysregulation of YAP is associated with a plethora of human cancers and is involved in cancer drug resistance (Nguyen and Yi (2019) TRENDS in CANCER 5:283-296; Zanconato et al. (2016) Cancer Cell 29:783-803). The IDR of YAP is required for LLPS and YAP condensate formation upon osmotic stress induced by sorbitol (Cai et al. (2019) Nat Cell Biol, 1-25).

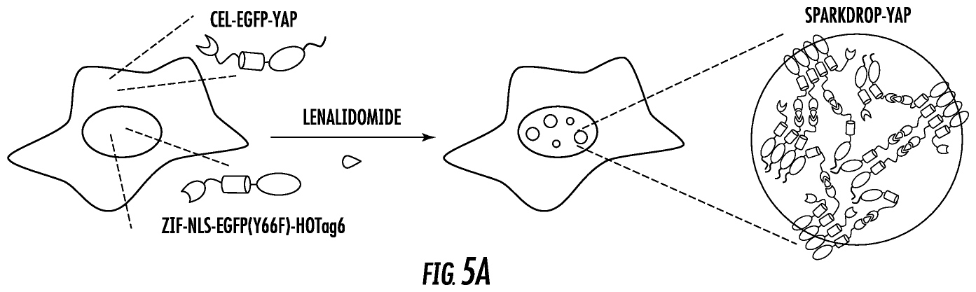

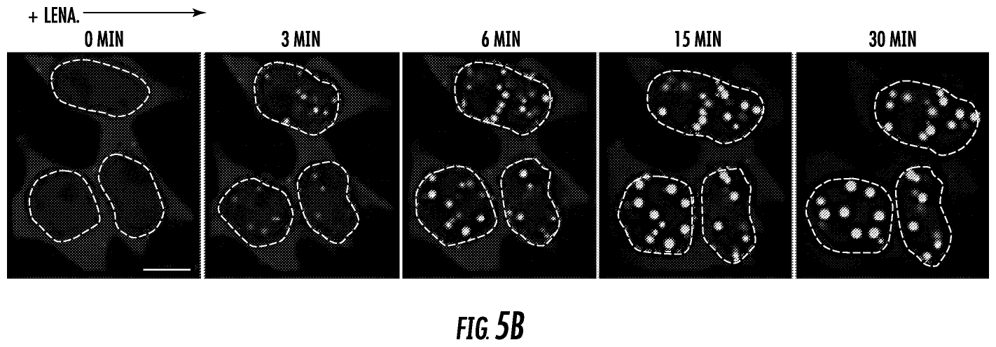

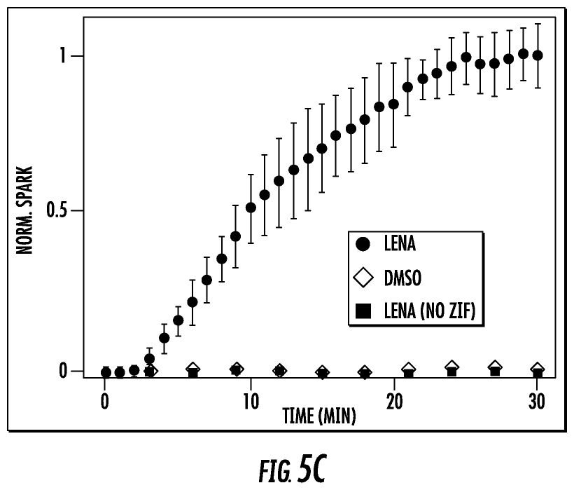

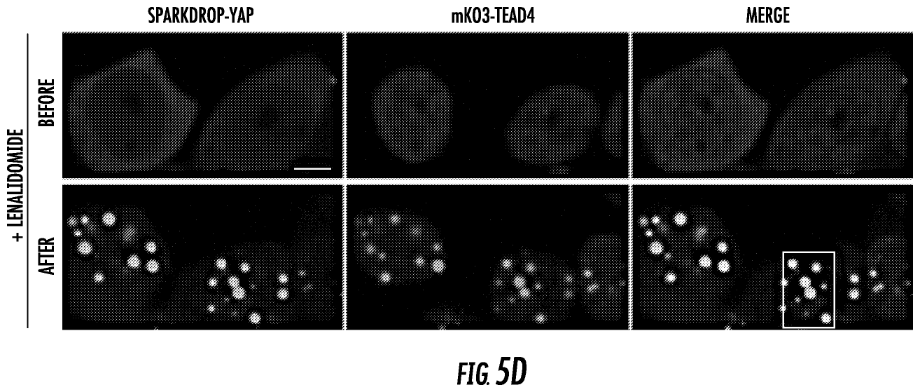

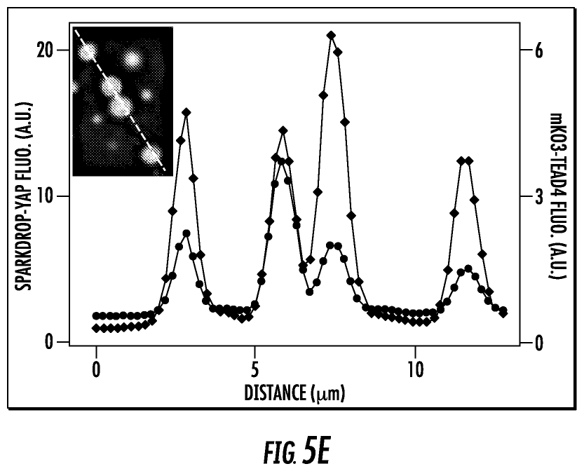

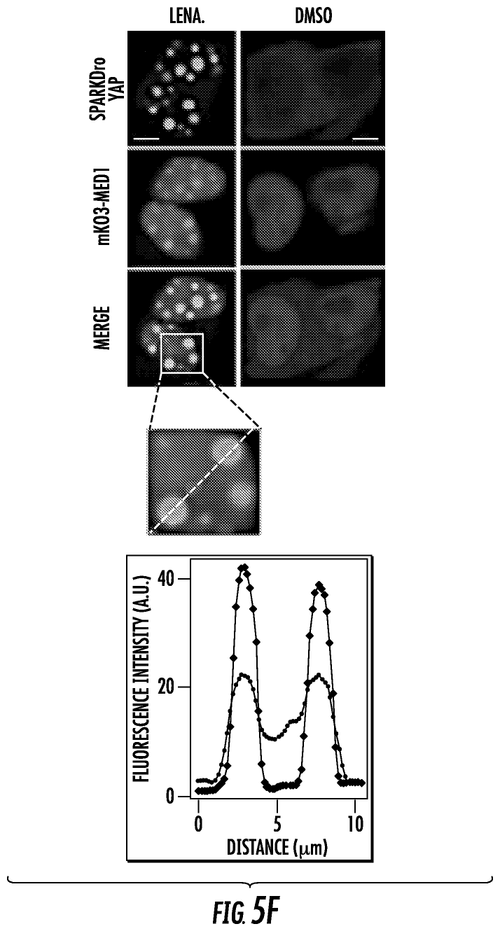

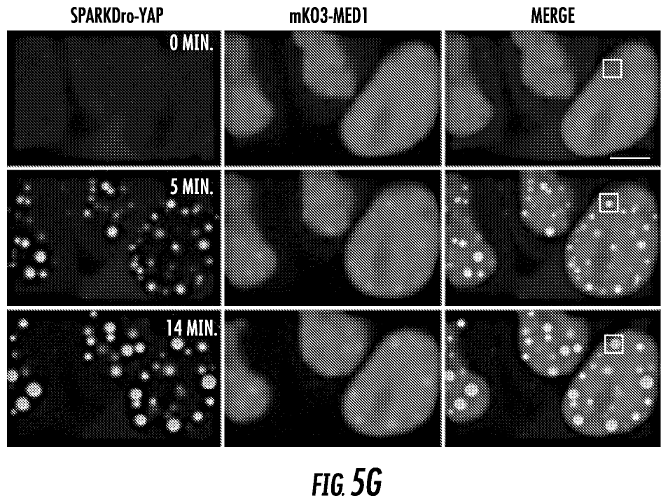

To induce nuclear condensates of full-length YAP, SparkDrop was applied to manipulate YAP phase separation and condensate formation without stimulating the Hippo pathway such as by sorbitol (FIG. 5A). First, YAP was fused to CEL and EGFP, and the fusion protein was localized to the cytoplasm as expected for YAP in the inactive state (FIG. 5B). Second, ZIF-NLS-EGFP (Y66F)-HOTag6 was co-expressed in the nucleus by incorporating a nuclear localization sequence (NLS). Because lenalidomide induces CEL and ZIF interaction, it is expected that upon addition of lenalidomide, the YAP fusion protein will translocate to the nucleus, and form condensates via multivalent interactions (FIG. 5A). Indeed, after addition of lenalidomide, green fluorescent droplets quickly formed in the nucleus at −3 minutes, which grew larger, forming intense green droplets within 20 to 30 minutes, indicating that the SparkDrop induced YAP LLPS and condensate formation (FIG. 5B). Meanwhile, the green fluorescence in the cytoplasm was depleted (FIG. 5B). Quantitative analysis of the time-lapse fluorescence images revealed kinetics of YAP condensation with a half-maximal time value (T112)—10 minutes (FIG. 5C). Two control experiments showed that DMSO did not induce YAP condensate formation, and that lenalidomide alone (lack of ZIF) could not induce YAP phase separation (FIG. 5C).

Next, to determine whether the SparkDrop-based YAP condensates interact and colocalize with the TEAD transcriptional factors. TEAD4 that is tagged by a red fluorescent protein mKO3 was co-expressed. Fluorescence imaging showed that, before addition of lenalidomide, YAP was located in the cytoplasm, whereas TEAD4 was in the nucleus but no TEAD4 droplets were observed (FIG. 5D). After addition of lenalidomide, YAP formed green condensates in the nucleus and that TEAD4 formed red droplets in the nucleus. Furthermore, the YAP and TEAD4 condensates were co-localized with each other (FIG. 5D and FIG. 5E). These data indicate that the SparkDrop-based YAP condensates compartmentalize TEAD4 and have a potential to activate gene transcription.

e. Sparkdrop-Based YAP Condensates Compartmentalize Transcriptional Machinery

To determine whether the SparkDrop-based YAP condensates can activate gene transcription, whether these condensates recruit transcriptional machinery was examined. Because Mediator complex is required for gene transcription by RNA polymerase II (RNAPII), whether the SparkDrop-YAP condensates recruit Mediator of RNA polymerase II transcription subunit 1 (MEDI) by tagging MEDI with mK03 was evaluated first.

Fluorescence imaging showed that lenalidomide induced SparkDrop-Y AP condensates, which recruited and compartmentalized MEDI (FIG. 5F). In contrast, DMSO did not induce green droplets or red condensates. Furthermore, time-lapse imaging showed that MEDI was recruited to the YAP condensates at early time after addition of lenalidomide, and that over time when the YAP condensates grew larger, more MED1 proteins were recruited and compartmentalized to the YAP condensates (FIG. 5G and FIG. 5H).

Next, whether the SparkDrop-Y AP condensates contain active RNAPII was imaged. The cells were stained with antibodies against the RNAPII that was phosphorylated at Ser5 (S5P) at the c-terminal domain {Lu: 2018bh}. Fluorescence imaging revealed that in cells with the green YAP condensates, RNAPII-SSP formed red punctate structures, and that the red condensates were co-localized to the green YAP droplets (FIG. 51, insets 1, 3, and 4, and FIG. SJ). In contrast, in untransfected cells without green YAP condensates, no red punctate structures were observed (FIG. 51, inset 2). As controls, the SparkDrop condensates (without YAP) did not recruit or compartmentalize the interacting protein TEAD4, components of transcriptional machinery including MEDI and active RNAPII-SSP (Supporting FIG. S8). Taken together, these data indicate that the SparkDrop-Y AP condensates recruit transcriptional machinery.

f. Sparkdrop-YAP Condensates Produce Nascent RNA and Control Target Gene Expression

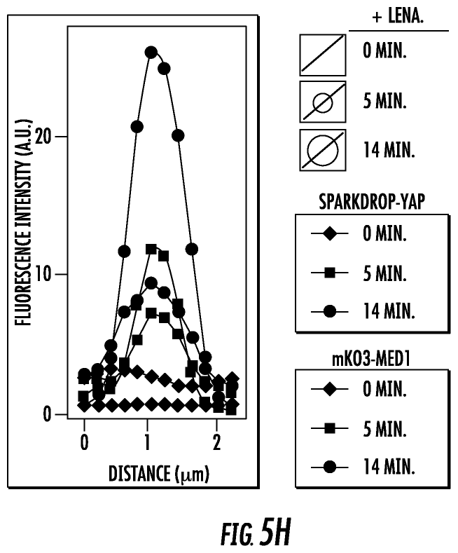

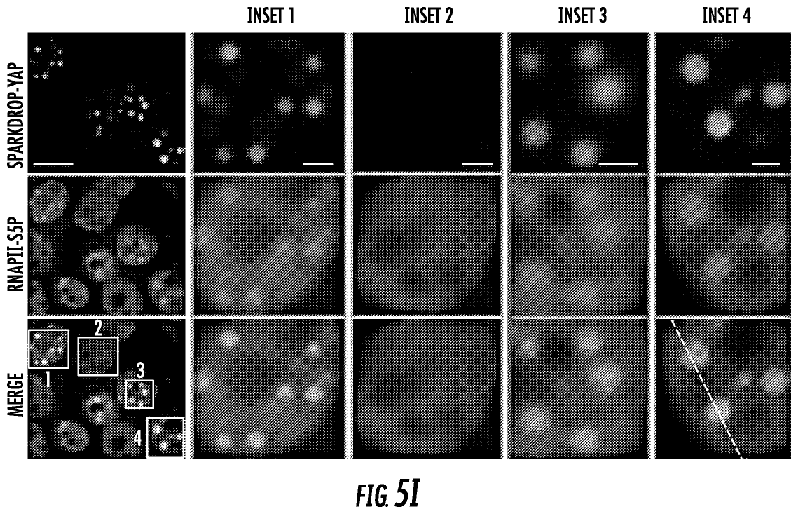

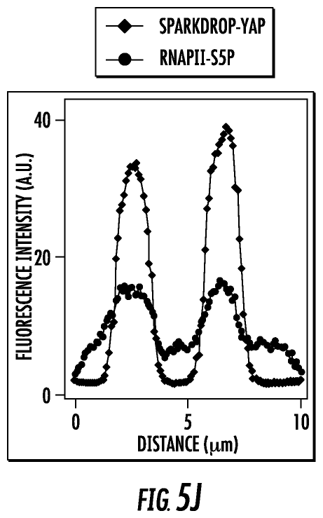

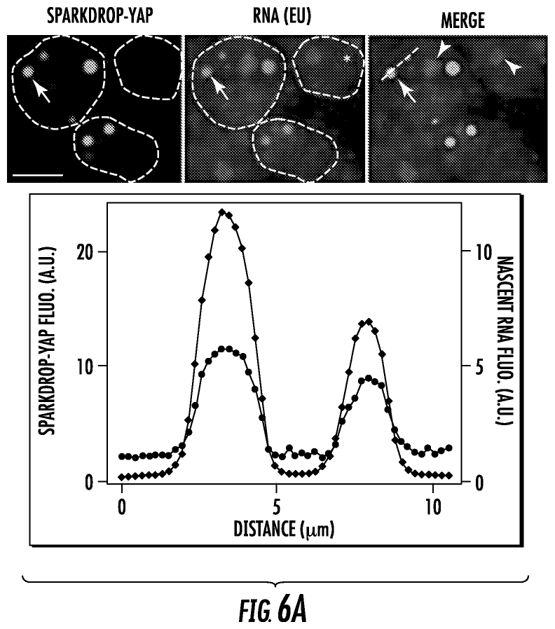

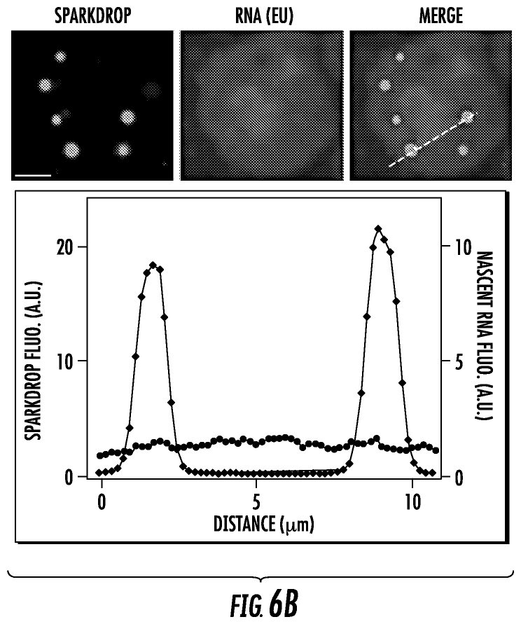

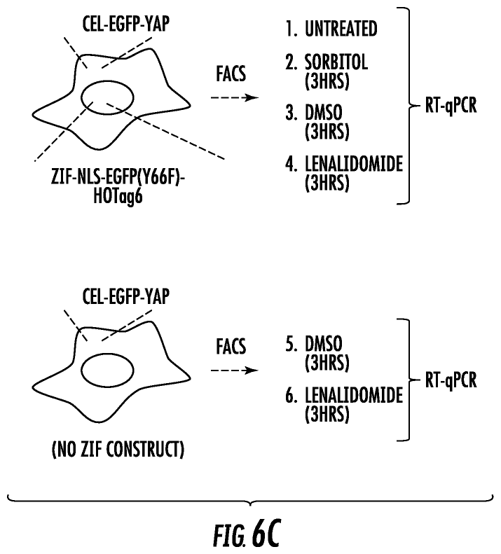

To finally determine whether the induced SparkDrop-YAP condensates are transcriptionally active, whether SparkDrop-Y AP condensates produce nascent RNAs was examined. The cells were incubated with uridine analog 5-ethynyluridine (EU) for 1 hour so that EU is incorporated it into newly transcribed RNA The EU-labeled nascent RNA is detected by using a copper (!)-catalyzed cycloaddition reaction (i.e., “click” chemistry) with azides labzeled with red fluorescent dyes. Fluorescence imaging in the red channel revealed several punctate structures (FIG. 6A). The round and relatively small structures of nascent RNAs were colocalized with the SparkDrop-Y AP condensates (FIG. 6A, arrows pointing to YAP condensates), suggesting that the SparkDrop-YAP condensates contain nascent RNAs. In the untransfected cells (FIG. 6A, asterisk-marked nucleus), the round and relatively small structures of nascent RNA were not present. Instead, large structures of nascent RNAs were observed (an arrowhead pointing to a nucleolus). These large structures are presumably nucleoli, sites of abundant ribosomal RNA transcription, which were reported to be stained with 5-EU (Jao eand Salic (2008) Proceedings of the National Academy of Sciences 105:15779-15784). Indeed, colocalization of these structures with NPMI-mEGFP was observed (Supporting FIG. S9). These results indicate that the induced SparkDrop-Y AP condensates are transcriptionally active and produce nascent RNAs. As a control, cells were transfected with SparkDrop constructs without YAP, incubated cells with lenalidomide to induce condensates. Nascent RNAs were labeled in these cells with EU, which showed that these YAP-absent SparkDrop condensates did not contain nascent RNAs (FIG. 6B).

Lastly, whether the SparkDrop-Y AP condensates upregulate mRNA levels of a YAP target gene Ctgf were determined using RT-qPCR. First, cells were transfected with both constructs of SparkDrop-Y AP, before being incubated with lenalidomide that induced SparkDrop-Y AP condensate formation. As a control, cells were incubated with DMSO. The data showed that SparkDrop-YAP condensates upregulated the Ctgf mRNA by −2.8-fold relative to that of the DMSO control (FIG. 6C and FIG. 6D). Next, a positive control was conducted by incubating cells with sorbitol, which is known to induce osmotic stress and drive YAP condensate formation. This upregulated Ctgf mRNA level by −4.5-fold relative to that of the untreated cells. Lastly, cells were transfected with CEL-EGFP-Y AP (without the ZIF construct), and incubated with lenalidomide or DMSO. The data indicate that Ctgf mRNA was not upregulated, suggesting that lenalidomide alone does not perturb Ctgf expression. Taken together, these results demonstrate that SparkDrop-YAP condensates are transcriptionally active, producing nascent RNAs in the condensates and upregulating mRNA levels of a direct target gene of YAP.

g. Discussion

Many biomolecular condensates have been discovered and investigated (Banani et al. (2017) Nat Rev Mol Cell Biol, 1-14; Shin and Brangwynne (2017) Science 357: eaaf4382; Lyon et al. (2021) Nat Rev Mol Cell Biol, 1-21; Alberti and Hyman (2021) Nat Rev Mol Cell Biol, 1-18; Boija (2021) Cancer Cell, 1-19; Harlen and Churchman (2017) Nat Rev Mol Cell Biol 18:263-273) since the ground-breaking work about a decade ago that reveals key roles of protein phase separation in formation of liquid-like condensates (Shin and Brangwynne (2017) Science 357: eaaf4382), and multivalent interactions that drive protein LLPS (Banani et al. (2017) Nat Rev Mol Cell Biol, 1-14). To further understand these biological condensates, including their assembly mechanisms and functional roles, versatile technologies are much needed to manipulate and decouple protein LLPS and condensate formation from biomolecular concentration and biological stimuli. Optogenetic tools have been demonstrated in manipulating protein phase separation and are greatly valuable in understanding biological condensates (Shin et al. (2018) Cell 175:1481-1491; Shin et al. (2016) Cell. 1-28: Wei et al. (2020) Nat Cell Biol, 1-19; Bracha et al. (2018) Cell 175:1467-1480.e13). Chemogenetic tools provide an orthogonal approach to manipulate LLPS and will also be valuable in investigating biological condensates by dissecting their assembly mechanisms and functional roles in cells.

In this work, a chemogenetic tool SparkDrop is designed that enables protein LLPS via small molecule-induced multivalent interactions. The operating physical principle of SparkDrop is consistent with one of the well-known biophysical driving forces of LLPS. i.e., multivalent PPis (Li et al. (2012) Nature 483:336-340). The SparkDrop condensates were characterized and found to be highly dynamic and possess liquid-like properties, likely resulting from the relatively weak interactions between CEL and ZIF that are inducible by lenalidomide. This PPI interaction is about one order of magnitude weaker than the rapamycin-inducible FKBP and Frb PPI pair (Banaszynski et al. (2005) J Am Chem Soc 127:4715-4721).

It has been demonstrated that SparkDrop is a versatile tool for dissecting assembly mechanisms of stress granules, suggesting that SparkDrop will be a useful technology in dissecting assembly mechanisms of protein condensates. Other than cytosolic condensates, SparkDrop has also been demonstrated in manipulating nuclear condensates. The SparkDrop-based YAP condensates recruit interacting proteins and components of transcriptional machinery including Mediator and active RNA polymerase II. Furthermore, they are transcriptionally active, producing nascent RNA in the synthetic YAP condensates and upregulating mRNA of a direct target gene of YAP. These data suggest that YAP condensates play a functional role in regulating gene transcription, consistent with previous studies (Cai et al. (2019) Nat Cell Biol. 1-25). The advantage of SparkDrop includes decoupling of YAP LLPS from osmotic stress, a stimulus of hippo pathway (Hong et al. (2017) EMBO Rep. 18:72-86). SparkDrop will thus be a valuable tool in understanding functional roles of nuclear condensates including condensates of many transcription factors (Lyon et al. (2021) Nat Rev Mol Cell Biol, 1-21; Sabari et al. (2020) Trends Biochem Sci 45:961-977; Sabari (2020) Dev Cell 55:84-96; Hnisz et al. (2017) Cell 169:13-2), which will illuminate and clarify the proposed phase separation model in regulation of gene transcription (Hnisz et al. (2017) Cell 169:13-23).

In summary, a chemogenetic tool, SparkDrop, has been designed and its application in dissecting assembly mechanisms and functional roles of cytosolic and nuclear condensates has been demonstrated. This small molecule-inducible tool provides an orthogonal approach to the light-inducible tools for manipulating LLPS of biomolecules and investigating condensates by decoupling LLPS from protein concentration and biological stimuli. Because of orthogonal approaches, this chemogenetic tool might be used together with the optogenetic tools for simultaneous manipulation of two protein LLPS. Furthermore, it has been demonstrated that SparkDrop can manipulate LLPS in a large number of cells, which allows for additional analysis, such as mRNA levels of a specific gene by RT-qPCR, to be performed. In the future, this tool may allow for chromatin conformation changes to be examined by technologies such as Hi-C. Therefore, together with the optogenetic tools, SparkDrop will be a valuable tool in dissecting biomolecular condensates for further understanding their roles in basic biology and disease.

### 2. Spark-Off: Dialing Down Phase Separation and Dissol Ving Condensates Using Highly Soluble Proteins

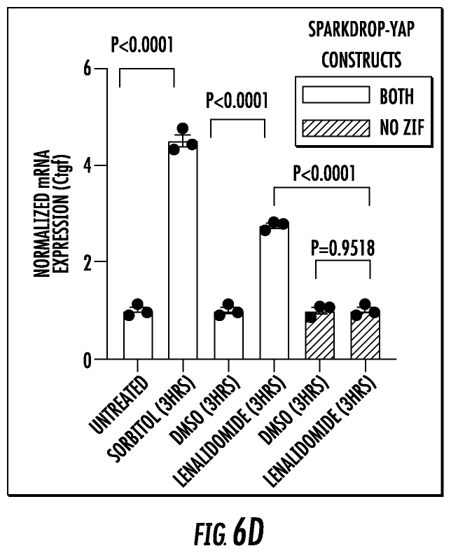

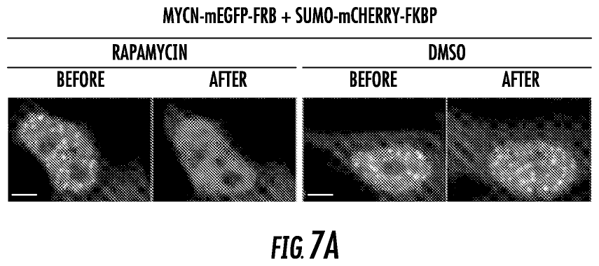

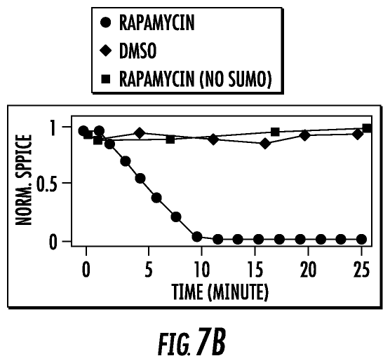

To dissolve the condensates while keeping the protein expression level constant, highly soluble proteins that effectively increase the critical concentration because of increased solvation by the aqueous medium were utilized. The highly soluble protein SUMO was initially chosen, as this protein is often used to improve solubility during protein purification. MYCN was fused to FRB and mEGFP and the fusion protein was expressed above the critical concentration so that it formed condensates. SUMO-mCherry-FKBP was co-expressed (see FIG. 1B). Fusing mCherry-FKBP to the c-terminus of SUMO also blocks its participation in the SUMOylation reaction. Rapamycin binds FKBP and induces its interaction with FRB, which recruits the highly soluble SUMO that dissolves the MYCN condensation in-10 min while keeping the MYCN expression unchanged (FIG. 7A). This result contrasts with the negative controls of DMSO and IFP2-mCherry-FKBP without SUMO (FIG. 7B), suggesting that SUMO-based dissolution of protein condensates is due to solubility rather than steric hindrance.

### H. REFERENCES

B. Alberts, The cell as a collection of protein machines: preparing the next generation of molecular biologists. Cell. 92, 291-294 (1998).
P. Li et al., Phase transitions in the assembly of multivalent signalling proteins. Nature. 483, 336-340 (2012).
C. P. Brangwynne et al., Germline P granules are liquid droplets that localize by controlled dissolution/condensation. Science. 324, 1729-1732 (2009).
S. F. Banani, H. O. Lee, A A Hyman, M. K. Rosen, Biomolecular condensates: organizers of cellular biochemistry. Nat Rev Mol Cell Biol, 1-14 (2017).
Y. Shin, C. P. Brangwynne, Liquid phase condensation in cell physiology and disease. Science. 357, eaaf4382 (2017).
L.-P. Bergeron-Sandoval, N. Safaee, S. W. Michnick, Perspective. Cell. 165, 1067-1079 (2016).
A A Hyman, C. A Weber, F. Julicher, Liquid-Liquid Phase Separation in Biology. Annu Rev Cell Dev Biol. 30, 39-58 (2014).
A S. Lyon, W. B. Peeples, M. K. Rosen, A framework for understanding the functions of biomolecular condensates across scales. Nat Rev Mol Cell Biol, 1-21 (2021).
S. Alberti, A A Hyman, Biomolecular condensates at the nexus of cellular stress, protein aggregation disease and ageing. Nat Rev Mol Cell Biol, 1-18 (2021).
Y. Shin et al., Liquid Nuclear Condensates Mechanically Sense and Restructure the Genome. Cell. 175, 1481-1491.el3 (2018).
Y. Shin et al., Spatiotemporal Control of Intracellular Phase Transitions Using Light-Activated optoDroplets. Cell, 1-28 (2016).
G. Lu et al., The myeloma drug lenalidomide promotes the cereblon-dependent destruction oflkaros proteins. Science. 343, 305-309 (2014).
G. Petzold, E. S. Fischer, N. H. Thoma, Structural basis of lenalidomide-induced CK1. Nature. 532, 127-130 (2016).
L. A Banaszynski, C. W. Liu, T. J. Wandless, Characterization of the FKBP.rapamycin.FRB ternary complex. J Am Chem Soc. 127, 4715-4721 (2005).
K. T. Siu et al., Preclinical activity of CPI-0610, a novel small-molecule bromodomain and extra-terminal protein inhibitor in the therapy of multiple myeloma. Leukemia. 31, 1760-1769 (2017).
M. E. Matyskiela et al., A novel cereblon modulator recruits GSPT1 to the CRL4. Nature. 535, 252-257 (2016).
S. Regot, J. J. Hughey, B. T. Bajar, S. Carrasco, M. W. Covert, High-Sensitivity Measurements of Multiple Kinase Activitiesin Live Single Cells. Cell. 157, 1724-1734 (2014).
Q. Zhang et al., Visualizing Dynamics of Cell Signaling In&nbsp; Vivo with a Phase Separation-Based Kinase Reporter. Mol Cell. 69, 334-345.e5 (2018).
M. Perie et al., Coexisting Liquid Phases Underlie Nucleolar Subcompartments. Cell. 165, 1686-1697 (2016).
C. P. Brangwynne, T. J. Mitchison, A A Hyman, Active liquid-like behavior of nucleoli determines their size and shape in Xenopus laevis oocytes. Proceedings of the National Academy of Sciences. 108, 4334-4339 (2011).
J. A Ditlev, L. B. Case, M. K. Rosen, J Mol Biol, in press, doi: 10.1016/j.jmb.2018.08.003.
S. F. Banani et al., Compositional Control of Phase-Separated Cellular Bodies. Cell. 166, 651-663 (2016).
R. J. Wheeler, A A Hyman, Controlling compartmentalization by non-membrane-bound organelles. Philosophical Transactions of the Royal Society B: Biological Sciences. 373, 20170193-9 (2018).
D. W. Sanders et al., Competing Protein-RNA Interaction Networks Control Multiphase Intracellular Organization. Cell. 181, 306-324.e28 (2020).
P. Yang et al., G3BP1 Is a Tunable Switch that Triggers Phase Separation to Assemble Stress Granules. Cell. 181, 325-345.e28 (2020).
J. Guillen-Boixet et al., RNA-Induced Conformational Switching and Clustering of G3BP Drive Stress Granule Assembly by Condensation. Cell. 181, 346-361.el 7 (2020).
N. Kedersha et al., G3BP-Caprin1-USP10 complexes mediate stress granule condensation and associate with 40S subunits. J Cell Biol. 212, 845-860 (2016).
S. Jain et al., ATPase-Modulated Stress Granules Contain a Diverse Proteome and Substructure. Cell. 164, 487-498 (2016).
I. M. Moya, G. Halder, Hippo-YAP/TAZ signalling in organ regeneration and regenerative medicine. Nat Rev Mol Cell Biol. 96, 1 (2018).
F.-X. Yu, Bin Zhao, K.-L. Guan, Hippo Pathway in Organ Size Control, Tissue Homeostasis, and Cancer. Cell. 163, 811-828 (2015).
A Pocaterra, P. Romani, S. Dupont, YAP/TAZ functions and their regulation at a glance. Journal of Cell Science. 133, jcs230425-9 (2020).
K. C. Lin, H. W. Park, K.-L. Guan, Regulation of the Hippo Pathway Transcription Factor TEAD. Trends Biochem Sci. 42, 862-872 (2017).
D. Cai et al., Phase separation of YAP reorganizes genome topology for long-term YAP target gene expression. Nat Cell Biol, 1-25 (2019).
S. A Manning, B. Kroeger, K. F. Harvey, The regulation of Yorkie, YAP and TAZ: new insights into the Hippo pathway. Development. 147, devl 79069-10 (2020).
C. D. K. Nguyen, C. Yi, YAP/TAZ Signaling and Resistance to Cancer Therapy. TRENDS in CANCER. 5, 283-296 (2019).
F. Zanconato, M. Cordenonsi, S. Piccolo, YAP/TAZ at the Roots of Cancer. Cancer Cell. 29, 783-803 (2016).
C. Y. Jao, A Salic, Exploring RNA transcription and turnover in vivo by using click chemistry. Proceedings of the National Academy of Sciences. 105, 15779-15784 (2008).
A Boija, Biomolecular condensates and cancer. Cancer Cell, 1-19 (2021).
K. M. Harlen, L. S. Churchman, The code and beyond: transcription regulation by the RNA polymerase II carboxy-terminal domain. Nat Rev Mol Cell Biol. 18, 263-273 (2017).
M.-T. Wei et al., Nucleated transcriptional condensates amplify gene expression. Nat Cell Biol, 1-19 (2020).
D. Bracha et al., Mapping Local and Global Liquid Phase Behavior in Living Cells Using Photo-Oligomerizable Seeds. Cell. 175, 1467-1480.el3 (2018).
A W. Hong et al., Osmotic stress-induced phosphorylation by NLK at Ser128 activates YAP. EMBO Rep. 18, 72-86 (2017).
B. R. Sabari, A Dall′Agnese, R. A Young, Biomolecular Condensates in the Nucleus. Trends Biochem Sci. 45, 961-977 (2020).
B. R. Sabari, Biomolecular Condensates and Gene Activation in Development and Disease. Dev Cell. 55, 84-96 (2020).
D. Hnisz, K. Shrinivas, R. A Young, A K. Chakraborty, P. A Sharp, Perspective. Cell. 169, 13-23 (2017).

It will be apparent to those skilled in the art that various modifications and variations can be made in the present invention without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A composition comprising:
a first amino acid sequence and a second amino acid sequence,
wherein the first amino acid sequence comprises a zinc finger domain, or a functional fragment thereof; and wherein the second amino acid sequence comprises a cereblon (CRBN) amino acid domain, or a functional fragment thereof.

2. The composition of claim 1, wherein the zinc finger is IZF1 or a functional fragment thereof.

3. The composition of claim 1, wherein the CRBN amino acid domain comprises at least 75% sequence identity to SEQ ID: 2.

4. The composition of claim 1 further comprising a third domain comprising a tag.

5.-6. (canceled)

7. The composition of claim 1, further comprising an inducer, wherein the first domain and the second domain are dimerized in the presence of an inducer.

8.-9. (canceled)

10. The composition of claim 1, wherein the first or second amino acid sequence further comprises a targeting domain; or wherein the first or second amino acid sequence is conjugated to a targeting domain by a linker.

11. The composition of claim 10, wherein the targeting domain is an antibody or antibody fragment comprising a first complementarity region (CDR) comprising a sequence that targets one or a plurality of cancer antigens with a Kd from about 0.1 nM to about 500 nM.

12.-14. (canceled)

15. The composition of claim 1 wherein the first or second amino acid domain further comprise an amino acid comprising one or a combination of: MYC, YAP, TAZ, YAP-MAML2, or a functional fragment thereof.

16. The composition of claim 1, wherein the composition is in the form of an amino acid granule that is free of a lipid membrane.

17. The composition of claim 16, wherein the granule is from about 0.1 micron to about 30 microns in width.

18. The composition of claim 1 further comprising a cell, the cell comprising the first and second amino acid sequences.

19. The composition of claim 18, wherein the cell further comprises an inducer with a concentration of from about 10 nM to about 10 μM in cytosol.

20. (canceled)

21. The composition of claim 18, wherein the cell is a cancer cell or a transformed cell.

22. The composition of claim 21, wherein the cell is a 293T cell or a NB cell.

23. A method of screening activity of plurality of compounds, the method comprising:
(a) exposing a compound to a composition comprising a first amino acid sequence
and a second amino acid sequence for a time period sufficient to induce disassociation or association of the first amino acid sequence from or to, respectively, the second amino acid sequence;
wherein the first amino acid sequence comprises a zinc finger domain, or a functional fragment thereof; and wherein the second amino acid sequence comprises a cereblon (CRBN) amino acid domain, or a functional fragment thereof.

24.-30. (canceled)

31. The method of claim 23, further comprising the step of exposing the first amino acid sequence to the second amino acid sequence to an inducer for a sufficient time period to induce association or dissociation of the first amino acid sequence to or from, respectively, the second amino acid sequence with each other, wherein such step is performed before step (a).

32. The method of claim 23, further comprising the step of measuring the amount of association or disassociation in the presence of one or a plurality of compounds as compared to the association or disassociation in the absence of one or a plurality of compounds; and characterizing the activity of the one or plurality of compounds based upon the magnitude of the binding.

33. The method of claim 23, wherein the first domain and second domain are dimerized at a concentration of each domain is from about 100 nM to about 900 nM in the presence of an inducer.

34.-35. (canceled)

36. A method of isolating a protein from a solution comprising:
(a) exposing a compound to a composition comprising a first amino acid sequence
and a second amino acid sequence for a time period sufficient to induce disassociation or association of the first amino acid sequence and the second amino acid sequence;
wherein the solution comprises a composition comprising: a first amino acid sequence and a second amino acid sequence: wherein the first amino acid sequence comprises a zinc finger domain, or a functional fragment thereof; and wherein the second amino acid sequence comprises a cereblon (CRBN) amino acid domain, or a functional fragment thereof.

37. The method of claim 36, wherein the step of exposing further comprises a step of associating or disassociating the first and second domains in the presence of an inducer.

38.-39. (canceled)

40. The composition of claim 1 further comprising:
a third amino acid sequence or a nucleic acid sequence encoding a third amino acid sequence:
wherein the first amino acid sequence comprises a zinc finger domain, or a functional fragment thereof; and wherein the second amino acid sequence comprises a cereblon (CRBN) amino acid domain, or a functional fragment thereof at a molecular ratio from about 1 to about 1; and
wherein the third amino acid sequence or nucleic acid sequence encoding the third amino acid sequence is encapsulated within a particle comprising the first and second amino acid sequence; and
wherein the third nucleic acid sequence encodes an enzyme or is an sgRNA.

41. (canceled)

42. A method of forming a particle in vivo or in vitro comprising:
(a) exposing a compound to a composition comprising a first amino acid sequence
and a second amino acid sequence for a time period sufficient to induce disassociation or association of the first amino acid sequence and the second amino acid sequence;
wherein the solution comprises a composition comprising: a first amino acid sequence and a second amino acid sequence,
wherein the first amino acid sequence comprises a zinc finger domain, or a functional fragment thereof; and wherein the second amino acid sequence comprises a cereblon (CRBN) amino acid domain, or a functional fragment thereof.

43. The method of claim 42, wherein the composition comprises an inverse fusion velocity or an inversion capillary velocity of from about 1 to about 10, in units of viscosity/units of surface tension.

44.-45. (canceled)

46. The method of claim 42, wherein the composition comprises an average inversion capillary from about 2.8 to about 3.3, in units of viscosity/units of surface tension.
